(19) United States
(12) Patent Application Publication
(10) Pub. No.: US 2009/0076062 A1
(43) Pub. Date: Mar. 19, 2009

(54) Organic Compounds

(76) Inventors: Juergen Klaus Maibaum, Weil-Haltingen (DE); Daniel Kaspar Baeschlin, Arlesheim (CH); Holger Sellner, Buchs (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/900,695

(22) Filed: Sep. 13, 2007

(51) Int. Cl. A61K 31/435; C07D 309/02; A61K 31/351; C07D 207/00; A61K 31/445; A61K 31/34; A61K 31/337; A61K 31/357; A61P 9/00; A61P 25/00; A61P 3/10; C07D 319/06; C07D 317/10; C07D 305/04; C07D 307/04; C07D 221/00; A61K 31/40; C07D 221/02

(52) U.S. Cl. 514/299; 549/424; 514/459; 548/557; 514/426; 546/112; 546/224; 514/329; 549/475; 514/472; 549/510; 514/449; 549/452; 514/467; 549/373; 514/452

(57) ABSTRACT

Disclosed are δ-amino-γ-hydroxy-ω-aryl-alkanoic acid amide compounds of formula (I)

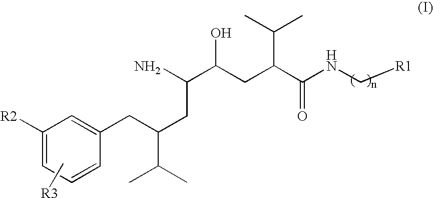

and the salts thereof, having renin-inhibiting properties. Also disclosed are pharmaceutical compositions comprising these compounds and methods of administering them for the treatment of hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders.

The invention relates to novel δ-amino-γ-hydroxy-ω-aryl-alkanoic acid amides of formula I, these compounds for use in the diagnostic and therapeutic treatment of a warm-blooded animal, especially for the treatment of a disease (=disorder) that depends on activity of renin; the use of a compound of that class for the preparation of a pharmaceutical formulation for the treatment of a disease that depends on activity of renin; the use of a compound of that class in the treatment of a disease that depends on activity of renin; pharmaceutical formulations comprising said novel δ-amino-γ-hydroxy-ω-aryl-alkanoic acid amide compound; a method of treatment comprising administering said novel δ-amino-γ-hydroxy-ω-aryl-alkanoic acid amides and a method for the manufacture of said novel δ-amino-γ-hydroxy-ω-aryl-alkanoic acid amide compounds.

### BACKGROUND OF THE INVENTION

We have described novel δ-amino-γ-hydroxy-ω-aryl-alkanoic acid amides which are useful as renin inhibitors (see, for example, U.S. Pat. No. 5,559,111). Although these compounds are suitable and effective for this purpose, there is a continued need to develop renin inhibitors with a further improved pharmacokinetic profile whilst at the same time achieving a good potency and safety profile. In particular, the provision of renin inhibitors with enhanced bioavailability is of therapeutic advantage. Bioavailability is an important factor limiting the therapeutic applications of bioactive compounds. The object of the present invention was thus to provide novel potent renin inhibitors with enhanced bioavailability.

### SUMMARY OF THE INVENTION

In one aspect, the invention relates to novel δ-amino-γ-hydroxy-ω-aryl-alkanoic acid amides of formula (I)

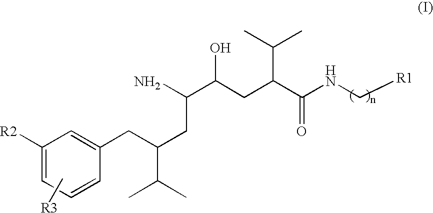

wherein

R1 is
a) oxetanyl, tetrahydrofuranyl, tetrahydropyranyl, furanyl, dioxolanyl or dioxanyl, each of which is optionally substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl, nitro, or oxo; or
b) piperidyl which is substituted, one or more times (e.g. one, two or three), by C₂₋₆alkenyl, C₂₋₆alkynyl, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl or nitro; which is bonded via a C atom; or
c) pyrrolidinyl which is substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro; which is bonded via a C atom; or
d) bicyclic saturated heterocyclyl which is optionally substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro; which is bonded via a C atom; and

R2 and R3, independently of one another, are selected from C₁₋₈alkyl, C₁₋₈alkoxy, C₁₋₄alkoxy-C₁₋₄alkoxy, C₁₋₄alkoxy-C₁₋₄alkyl, halo-C₁₋₈alkoxy, halo-C₁₋₈alkyl, C₁₋₈alkanoyl, C₃₋₈cycloalkyl or halogen; and

n is 0 or 1;

or a salt thereof.

In another aspect, the present invention also relates to a compound of the formula (II)

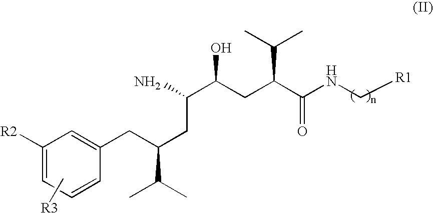

wherein

R1 is
a) oxetanyl, tetrahydrofuranyl, tetrahydropyranyl, furanyl, dioxolanyl or dioxanyl, each of which is optionally substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl, nitro, or oxo; or
b) piperidyl which is substituted, one or more times (e.g. one, two or three), by C₂₋₆alkenyl, C₂₋₆alkynyl, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl or nitro; which is bonded via a C atom; or
c) pyrrolidinyl which is substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro; which is bonded via a C atom; or
d) bicyclic saturated heterocyclyl which is optionally substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro; which is bonded via a C atom; and

R2 and R3, independently of one another, are selected from C₁₋₈alkyl, C₁₋₈alkoxy, C₁₋₄alkoxy-C₁₋₄alkoxy, C₁₋₄alkoxy-C₁₋₄alkyl, halo-C₁₋₈alkoxy, halo-C₁₋₆alkyl, C₁₋₈alkanoyl, C₃₋₈cycloalkyl or halogen; and

n is 0 or 1;

or a salt thereof.

In a further aspect, the present invention also relates to a compound of the formula (III)

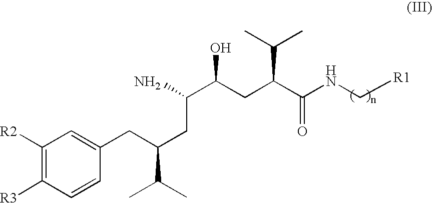

wherein

R1 is
a) oxetanyl, tetrahydrofuranyl, tetrahydropyranyl, furanyl, dioxolanyl or dioxanyl, each of which is optionally substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl, nitro, or oxo; or
b) piperidyl which is substituted, one or more times (e.g. one, two or three), by C₂₋₆alkenyl, C₂₋₆alkynyl, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl or nitro; which is bonded via a C atom; or
c) pyrrolidinyl which is substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro; which is bonded via a C atom; or
d) bicyclic saturated heterocyclyl which is optionally substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro; which is bonded via a C atom; and

R2 and R3, independently of one another, are selected from C₁₋₆alkyl, C₁₋₈alkoxy, C₁₋₄alkoxy-C₁₋₄alkoxy, C₁₋₄alkoxy-C₁₋₄alkyl, halo-C₁₋₈alkoxy, halo-C₁₋₈alkyl, C₁₋₈alkanoyl, C₃₋₈cycloalkyl or halogen; and

n is 0 or 1;

or a salt thereof.

In a still further aspect, the present invention also relates to a compound of the formula (IV)

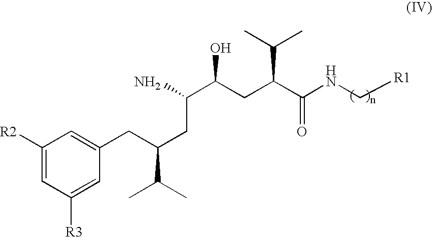

wherein

R1 is
a) oxetanyl, tetrahydrofuranyl, tetrahydropyranyl, furanyl, dioxolanyl or dioxanyl, each of which is optionally substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl, nitro, or oxo; or
b) piperidyl which is substituted, one or more times (e.g. one, two or three), by C₂₋₆alkenyl, C₂₋₆alkynyl, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl or nitro; which is bonded via a C atom; or
c) pyrrolidinyl which is substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro; which is bonded via a C atom; or
d) bicyclic saturated heterocyclyl which is optionally substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro; which is bonded via a C atom, and

R2 and R3, independently of one another, are selected from C₁₋₆alkyl, C₁₋₆alkoxy, C₁₋₄alkoxy-C₁₋₄alkoxy, C₁₋₄alkoxy-C₁₋₄alkyl, halo-C₁₋₈alkoxy, halo-C₁₋₈alkyl, C₁₋₈alkanoyl, C₃₋₈cycloalkyl or halogen; and

n is 0 or 1;

or a salt thereof.

According to the present invention, there are provided chemical compounds of the formulae (I), (II), (III) and (IV), or salts thereof, preferably pharmaceutically acceptable salts thereof, to processes for the preparation of the compounds according to the invention, to pharmaceutical compositions containing them and to their use as medicinal active ingredients.

The compounds of the present invention have enzyme-inhibiting properties. In particular, they inhibit the action of the natural enzyme renin. The latter passes from the kidneys into the blood where it effects the cleavage of angiotensinogen, releasing the decapeptide angiotensin I which is then cleaved in the lungs, the kidneys and other organs to form the octapeptide angiotensinogen II. The octapeptide increases blood pressure both directly by arterial vasoconstriction and indirectly by liberating from the adrenal glands the sodium-ion-retaining hormone aldosterone, accompanied by an increase in extracellular fluid volume. That increase can be attributed to the action of angiotensin II. Inhibitors of the enzymatic activity of renin bring about a reduction in the formation of angiotensin I. As a result a smaller amount of angiotensin II is produced. The reduced concentration of that active peptide hormone is the direct cause of the hypotensive effect of renin inhibitors.

Thus, the compounds of the present invention may be employed for the treatment of hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders.

Listed below are definitions of various terms used to describe the compounds of the present invention. These definitions apply to the terms as they are used throughout the specification unless they are otherwise limited in specific instances either individually or as part of a larger group.

Hereinbefore and hereinafter, lower radicals and compounds are to be understood as being, e.g., those having up to and including 7 carbon atoms, preferably up to and including 4 carbon atoms.

The term “C₁₋₈” defines a moiety with up to and including maximally 7, especially up to and including maximally 4, carbon atoms, said moiety being branched (one or more times) or straight-chained and bound via a terminal or a non-terminal carbon.

The term C₁₋₆alkyl defines a moiety with up to and including maximally 6, especially up to and including maximally 4, carbon atoms, said moiety being branched (one or more times) or straight-chained and bound via a terminal or a non-terminal carbon. C₁-C₆-alkyl, for example, is n-pentyl or n-hexyl or preferably C₁-C₄-alkyl, especially as methyl, ethyl, n-propyl, sec-propyl, n-butyl, isobutyl, sec-butyl or tert-butyl.

C₂-C₆alkenyl may be straight-chain or branched and is, for example, vinyl or allyl.

C₂₋₆alkynyl may be straight-chain or branched and is, for example, ethynyl.

C₁₋₆alkoxy may be linear or branched. Examples are methoxy, ethoxy, n- and i-propyloxy, n-, i- and t-butyloxy, pentyloxy and hexyloxy; C₁-C₄alkoxy is preferred. Most preferred is methoxy.

C₁₋₆alkoxy-C₁₋₆alkoxy may be linear or branched. The alkoxy group preferably comprises 1 to 4 C atoms. Examples are methoxymethyloxy, 2-methoxyethyloxy, 3-methoxypropyloxy, 4-methoxybutyloxy, 5-methoxypentyloxy, 6-methoxyhexyloxy, ethoxymethyloxy, 2-ethoxyethyloxy, 3-ethoxypropyloxy, 4-ethoxybutyloxy, 5-ethoxypentyloxy, 6-ethoxyhexyloxy, propyloxymethyloxy, butyloxymethyloxy, 2-propyloxyethyloxy and 2-butyloxyethyloxy. Preferred is 3-methoxypropyloxy.

C₁₋₆alkoxy-C₁₋₆alkyl may be linear or branched. The alkoxy group preferably comprises 1 to 4 and especially 1 or 2 C atoms, and the alkyl group preferably comprises 1 to 4 C atoms. Examples are methoxymethyl, 2-methoxyethyl, 3-methoxypropyl, 4-methoxybutyl, 5-methoxypentyl, 6-methoxyhexyl, ethoxymethyl, 2-ethoxyethyl, 3-ethoxypropyl, 4-ethoxybutyl, 5-ethoxypentyl, 6-ethoxyhexyl, propyloxymethyl, butyloxymethyl, 2-propyloxyethyl and 2-butyloxyethyl. Preferred is 4-methoxybutyl.

C₁₋₆alkoxycarbonylamino is, e.g., methoxycarbonylamino, ethoxycarbonylamino, propyloxycarbonylamino, isobutyloxycarbonylamino, butyloxycarbonylamino, isobutyloxycarbonylamino, secondary butyloxycarbonylamino or tertiary butyloxyamino.

C₀₋₆alkylcarbonylamino is, for example, formylamino, acetylamino, propionylamino, propylcarbonylamino, isopropylcarbonylamino, butylcarbonylamino, isobutylcarbonylamino, secondary butylcarbonylamino, tertiary butylcarbonylamino, pentylcarbonylamino or hexylcarbonylamino.

C₁₋₆alkylcarbonyloxy is, for example, acetyloxy, propionyloxy, propylcarbonyloxy, isopropylcarbonyloxy, butylcarbonyloxy, isobutylcarbonyloxy, secondary butylcarbonyloxy, tertiary butylcarbonyloxy, pentylcarbonyloxy or hexylcarbonyloxy.

C₁₋₆alkylenedioxy is, for example, methylenedioxy, ethylenedioxy, 1,3-propylelnedioxy or 1,2-propylenedioxy.

Optionally N-mono or N,N-di-C₁₋₆alkylated amino is, for example, dimethylamino, methylamino, N-methyl-N-ethylamino, ethylamino, diethylamino, N-methyl-N-propylamino, propylamino, butylamino or N-butyl-N-methylamino.

Optionally N-mono or N,N-di-C₁₋₆alkylated carbamoyl, is, for example, carbamoyl, methylcarbamoyl, ethylcarbamoyl, N,N-dimethylcarbamoyl, N,N-diethylcarbamoyl or propylcarbamoyl.

Optionally esterified carboxy is, for example, carboxy esterified with C₀₋₆alkyl, such as carboxy or C₁₋₆alkoxycarbonyl.

C₃₋₈cycloalkyl and C₃₋₈cycloalkyl in C₃₋₈cycloalkyl-C₀₋₆alkyl refers to cyclic hydrocarbon groups of 3 to 8 carbon atoms; preferably 3-, 5- or 6-membered cycloalkyl such as cyclopropyl, cyclopentyl and cyclohexyl.

C₃₋₈cycloalkoxy is, e.g., 3- to 8-membered, preferably 3-, 5- or 6-membered, cycloalkoxy, such as cyclopropyloxy, cyclopentyloxy, cyclohexyloxy, also cyclobutyloxy, cycloheptyloxy or cyclooctyloxy.

Halo or halogen is preferably fluoro, chloro, bromo or iodo, most preferably fluoro, chloro or bromo.

halo-C₁₋₆alkoxy is, for example, alkoxy substituted one or more (e.g. one, two or three) times by fluorine, chlorine, bromine or iodine, including mixed, e.g. fluorine and chlorine, substitutions, with preference for periluorinated radicals such as trifluoromethoxy. Preferred is halo-C₁₋₄alkoxy.

halo-C₁₋₆alkyl is in particular halo-C₁-C₄alkyl, such as trifluoromethyl, 1,1,2-trifluoro-2-chloroethyl or chloromethyl. Preferred halo-C₁-C₆alkyl is trifluoromethyl.

C₁₋₈alkanoyl and is, for example, formyl, acetyl [—C(═O)Me], propionyl, butyryl, isobutyryl or pivaloyl.

Heterocyclyl is a mono- or polycyclic, preferably a mono-, bi- or tricyclic-, most preferably mono-, unsaturated, partially saturated, saturated or aromatic ring system with preferably 3 to 22 (more preferably 3 to 14, most preferably 5 to 10) ring atoms and with one or more, preferably one to four, heteroatoms independently selected from nitrogen, oxygen, sulfur, S(═O)— or S—(═O)₂. When the heterocyclyl is an aromatic ring system, it is also referred to as heteroaryl. Preferred heterocyclic radicals are 3-8 membered, particularly preferably 5 or 6 membered, having 1, 2, 3 or 4 hetero atoms selected from the group consisting of N, S and O and are monocyclic and are optionally fused to a 3-8-membered ring which may be carbocyclic or heterocyclic. A further preferred group of heterocyclic radicals are bicyclic heterocycles which have a spiro-cyclic or bridged ring. Preferred heterocyclic radicals have in one or each ring 1 nitrogen, oxygen or sulphur atom, 1-2 nitrogen atoms, 1-2 oxygen atoms or 1-2 nitrogen atoms and 1-2 sulphur atoms, with at least one, preferably 1-7, carbon atom(s) being present in each ring. A preferred group of bicyclic heterocycles are saturated and have 1 nitrogen atom.

Aryl and aryl in aryl-C₁₋₆alkyl, aryl-C₁₋₆alkoxy and the like is preferably a mono- or bicyclic aryl with 6 to 22 carbon atoms, especially phenyl, indenyl, indanyl or naphthyl, in particular phenyl.

The symbol “*” defines a bond connecting the respective moiety to the rest of the molecule. Depending on the presence of asymmetric carbon atoms, the compounds of the invention may be in the form of mixtures of isomers, specifically as racemates, or in the form of pure isomers, specifically of optical antipodes. The invention includes all these forms. Mixtures of diastereomers, diastereomeric racemates or mixtures of diastereomeric racemates can be fractionated by conventional methods, e.g. by column chromatography, thin-layer chromatography, HPLC and the like.

Salts of compounds with salt-forming groups are in particular acid addition salts, salts with bases or, if a plurality of salt-forming groups is present, optionally also mixed salts or inner salts.

Salts are primarily the pharmaceutically acceptable or non-toxic salts of compounds of the formula (I). Such salts are formed for example by compounds of the formula (I) having an acidic group, e.g. a carboxy or sulpho group, and are for example their salts with suitable bases, such as non-toxic metal salts derived from metals of group Ia, Ib, IIa and IIb of the Periodic Table of the Elements, e.g. alkali metal, in particular lithium, sodium or potassium; salts, alkaline earth metal salts, for example magnesium or calcium salts, furthermore zinc salts or ammonium salts, also salts formed with organic amines such as optionally hydroxy-substituted mono-, di- or trialkylamines, especially mono-, di- or tri-lower-alkylamines, or with quaternary ammonium bases, e.g. methyl-, ethyl-, diethyl- or triethylamine, mono-, bis- or tris(2-hydroxy-lower-alkyl) amines such as ethanol-, diethanol- or triethanolamine, tris(hydroxymethyl)methylamine or 2-hydroxy-tertiary-butylamine, N. N-di-lower-alkyl-N-(hydroxy-lower-alkyl)amine, such as N,N-dimethyl-N-(2-hydroxyethyl)amine, or N-methyl-D-glucamine, or quaternary ammonium hydroxides such as tetrabutylammonium hydroxide.

The compounds of the formula I having a basic group, e.g. an amino group, can form acid addition salts, e.g. with suitable inorganic acids, e.g. hydrohalic acid such as hydrochloric acid, hydrobromic acid, sulphuric acid with replacement of one or both protons, phosphoric acid with replacement of one or more protons, e.g. orthophosphoric acid or metaphosphoric acid, or pyrophosphoric acid with replacement of one or more protons, or with organic carboxylic, sulphonic or phosphonic acids or N-substituted sulphamic acids, e.g. acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid, isonicotinic acid, furthermore amino acids such as, for example, the a-amino acids mentioned hereinbelow, and methanesuiphonic acid, ethanesuiphonic acid, 2-hydroxyethanesulphonic acid, ethane-1,2-disulphonic acid, benzenesulphonic acid, 4-toluenesuiphonic acid, naphthalene-2-suiphonic acid, 2- or 3-phosphoglycerate, glucose 6-phosphate, N-cyclohexylsulphamic acid (to form cyclamates) or with other acidic organic compounds such as ascorbic acid.

Compounds of the formula (I) having acidic and basic groups may also form inner salts.

Pharmaceutically unsuitable salts may also be used for isolation and purification.

The compounds of the formula (I) also include compounds in which one or more atoms are replaced by their stable, non-radioactive isotopes; for example a hydrogen atom by deuterium.

Prodrug derivatives of the compounds described herein are derivatives thereof which on in vivo use liberate the original compound by a chemical or physiological process. A prodrug may for example be converted into the original compound when a physiological pH is reached or by enzymatic conversion. Possible examples of prodrug derivatives are esters of freely available carboxylic acids, S- and 0-acyl derivatives of thiols, alcohols or phenols, the acyl group being defined as above. Preferred derivatives are pharmaceutically acceptable ester derivatives which are converted by solvolysis in physiological medium into the original carboxylic acid, such as, for example, lower alkyl esters, cycloalkyl esters, lower alkenyl esters, benzyl esters, mono- or disubstituted lower alkyl esters such as lower o-(amino, mono- or dialkylamino, carboxy, lower alkoxycarbonyl)-alkyl esters or such as lower a-(alkanoyloxy, alkoxycarbonyl or dialkylaminocarbonyl)-alkyl esters; conventionally, pivaloyloxymethyl esters and similar esters are used as such.

Because of the close relationship between a free compound, a prodrug derivative and a salt compound, a particular compound in this invention also includes its prodrug derivative and salt form, where this is possible and appropriate.

### PREFERRED EMBODIMENTS ACCORDING TO THE INVENTION

The groups of compounds mentioned below are not to be regarded as exclusive; rather, e.g., in order to replace general definitions with more specific definitions, parts of those groups of compounds can be interchanged or exchanged for the definitions given above, or omitted, as appropriate.

### Preferred Definitions for R1 and N

In a first embodiment, preferred are the compounds of the formulae (I), (II), (III) and (IV), or salts thereof, wherein R1 is selected from oxetanyl, tetrahydrofuranyl, tetrahydropyranyl, furanyl, dioxolanyl or dioxanyl, each of which is optionally substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl, nitro, or oxo; preferably wherein R1 is oxetanyl, tetrahydrofuranyl, tetrahydropyranyl, furanyl, dioxolanyl or dioxanyl, each of which is optionally substituted, one or more times (e.g. one, two or three), by C₁₋₆alkoxy, C₁₋₆alkyl, aryl-C₁₋₆alkyl, optionally esterified carboxy, C₃₋₈cycloalkyl-C₀₋₆alkyl, hydroxyl or oxo; more preferably wherein R1 is oxetanyl, tetrahydrofuranyl, tetrahydropyranyl, furanyl, dioxolanyl or dioxanyl, each of which is optionally substituted, one or more times (e.g. one, two or three), by C₁₋₆alkyl, aryl-C₁₋₆alkyl, optionally esterified carboxy, hydroxyl or oxo; most preferably wherein R1 is oxetanyl, tetrahydrofuranyl, tetrahydropyranyl, furanyl, dioxolanyl or dioxanyl, each of which is optionally substituted, one or more times (e.g. one, two or three), by C₁₋₆alkyl, hydroxyl or oxo.

In a particular embodiment thereof, preferred are the compounds of the formulae (I), (II), (III) and (IV), or salts thereof, wherein R1 is selected from oxetanyl, tetrahydrofuranyl, tetrahydropyranyl, furanyl, dioxolanyl or dioxanyl, each of which is unsubstituted.

In a particular embodiment thereof, preferred are the compounds of the formulae (I), (II), (III) and (IV), or salts thereof, wherein R1 is selected from oxetanyl, tetrahydrofuranyl, tetrahydropyranyl, furanyl, dioxolanyl or dioxanyl, each of which is substituted, one or more times (e.g. one, two or three), by methyl or hydroxyl.

In a particular embodiment thereof, preferred are the compounds of the formulae (I), (II), (III) and (IV), or salts thereof, wherein *-(CH₂)ₙR1 is selected from the group consisting of:

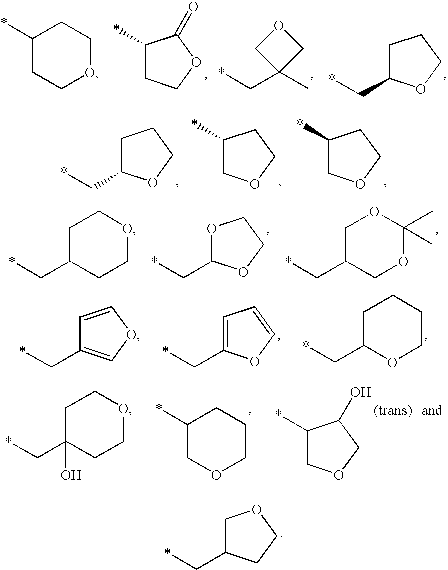

Particularly preferred are compounds of (III) wherein *-(CH₂)ₙR1 is selected from the group consisting of:

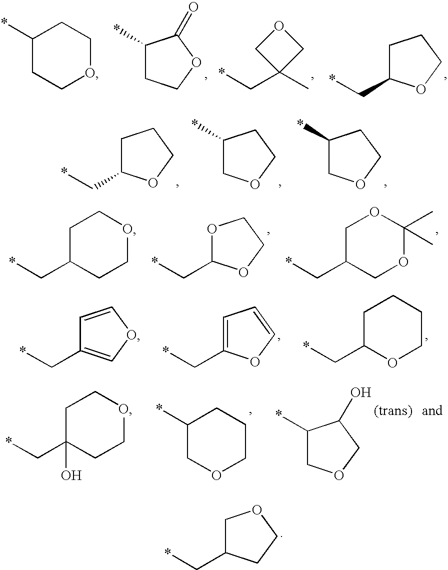

Also particularly preferred are compounds of (IV) wherein *-(CH₂)ₙR1 is selected from the group consisting of:

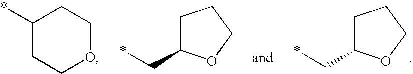

In a second embodiment, preferred are the compounds of the formulae (I), (II), (III) and (IV), or salts thereof, wherein R1 is piperidyl which is substituted, one or more times (e.g. one, two or three), by C₂₋₆alkenyl, C₂₋₆alkynyl, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl or nitro, which is bonded via a C atom; preferably wherein R1 is piperidyl which is substituted, one or more times (e.g. one, two or three), by C₁₋₆alkyl, aryl-C₁₋₆alkyl, optionally esterified carboxy or C₃₋₈cycloalkyl-C₀₋₆alkyl; which is bonded via a C atom; more preferably wherein R1 is piperidyl which is substituted, one or more times (e.g. one, two or three), by C₁₋₆alkyl, aryl-C₁₋₆alkyl or optionally esterified carboxy; which is bonded via a C atom. In this embodiment, n is preferably 0.

In a third embodiment, preferred are the compounds of the formulae (I), (II), (III) and (IV), or salts thereof, wherein R1 is pyrrolidinyl, which is substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro, which is bonded via a C atom; preferably wherein R1 is pyrrolidinyl, which is substituted, one or more times (e.g. one, two or three), by C₁₋₆alkoxy, C₁₋₆alkyl, aryl-C₁₋₆alkyl, optionally esterified carboxy, C₃₋₈cycloalkyl-C₀₋₆alkyl or hydroxyl; which is bonded via a C atom; more preferably wherein R1 is pyrrolidinyl, which is substituted, one or more times (e.g. one, two or three), by C₁₋₆alkyl, aryl-C₁₋₆alkyl, optionally esterified carboxyor hydroxyl; which is bonded via a C atom. In this embodiment, n is preferably 0.

In a forth embodiment, preferred are the compounds of the formulae (I), (II), (III) and (IV), or salts thereof, wherein R1 is bicyclic saturated heterocyclyl which is optionally substituted, one or more times (e.g. one, two or three), by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino,

C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl, nitro, or oxo; preferably wherein R1 is bicyclic saturated heterocyclyl which is optionally substituted, one or more times (e.g. one, two or three), by C₁₋₆alkoxy, C₁₋₆alkyl, aryl-C₁₋₆alkyl, optionally esterified carboxy, C₃₋₈cycloalkyl-C₀₋₆alkyl, hydroxyl or oxo; more preferably wherein R1 is bicyclic saturated heterocyclyl which is optionally substituted, one or more times (e.g. one, two or three), by C₁₋₆alkyl, aryl-C₁₋₆alkyl, optionally esterified carboxy, hydroxyl or oxo. In a preferred embodiment the bicyclic saturated heterocyclic radical is unsubstituted and comprises a nitrogen atom as heretoatom. In this embodiment, n is preferably 0.

In a particular embodiment of the second to forth embodiments, preferred are the compounds of the formulae (I), (II), (III) and (IV), or salts thereof, wherein *-(CH₂)ₙR1 is selected from the group consisting of:

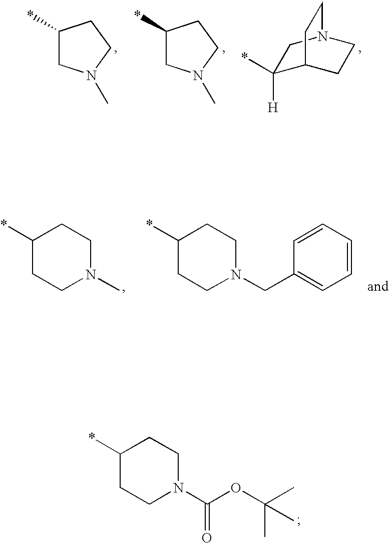

more preferably compounds of formula (III) or salts thereof.

### Preferred Definitions for R2 and R3

In an embodiment, preferred are the compounds of the formulae (I), (II), (III) and (IV), or salts thereof, wherein R2 is selected from C₁₋₆alkyl, C₁₋₈alkoxy, C₁₋₄alkoxy-C₁₋₄alkoxy or C₁₋₄alkoxy-C₁₋₄alkyl; preferably C₁₋₄alkoxy-C₁₋₄alkoxy or C₁₋₄alkoxy-C₁₋₄alkyl; most preferably 3-methoxypropyloxy, 2-methoxyethyloxy or 4-methoxybutyl.

In another embodiment, preferred are the compounds of the formulae (I), (II), (III) and (IV), or salts thereof, wherein R3 is selected from C₁₋₆alkyl, C₁₋₈alkoxy, C₁₋₄alkoxy-C₁₋₄alkoxy or C₁₋₄alkoxy-C₁₋₄alkyl; preferably C₁₋₈alkyl or C₁₋₈alkoxy; most preferably methyl or methoxy.

Particularly preferred embodiments are the compounds of the formulae (I), (II), (III) and (IV), or salts thereof, wherein R2 is C₁₋₄alkoxy-C₁₋₄alkoxy and R3 is C₁₋₆alkyl or C₁₋₈alkoxy; preferably wherein R2 is 3-methoxypropyloxy or 2-methoxyethyloxy and R3 is C₁₋₈alkyl or C₁₋₈alkoxy; more preferably wherein R2 is C₁₋₄alkoxy-C₁₋₄alkoxy and R3 is methyl or methoxy; still more preferably wherein R2 is 3-methoxypropyloxy or 2-methoxyethyloxy and R3 is methoxy or methyl; yet more preferably wherein R2 is 2-methoxyethyloxy and R3 is methoxy; even more preferably wherein R2 is 3-methoxypropyloxy and R3 is methoxy or methyl; most preferably wherein R2 is 3-methoxypropyloxy and R3 is methoxy.

Also particularly preferred embodiments are the compounds of the formulae (I), (II), (III) and (IV), or salts thereof, wherein R2 is C₁₋₄alkoxy-C₁₋₄alkyl and R3 is C₁₋₈alkyl or C₁₋₈alkoxy; preferably wherein R2 is C₁₋₄alkoxy-C₁₋₄alkyl and R3 is C₁₋₈alkoxy; more preferably wherein R2 is C₁₋₄alkoxy-C₁₋₄alkyl and R3 is methoxy; even more preferably wherein R2 is 4-methoxybutyl and R3 is C₁₋₈alkoxy; most preferably wherein R2 is 4-methoxybutyl and R3 is methoxy.

In one embodiment, preferred are the compounds of the formula (III), or salts thereof, wherein R2 is C₁₋₄alkoxy-C₁₋₄alkoxy and R3 is C₁₋₈alkyl or C₁₋₈alkoxy; preferably wherein R2 is 3-methoxypropyloxy or 2-methoxyethyloxy and R3 is C₁₋₈alkyl or C₁₋₈alkoxy; more preferably wherein R2 is C₁₋₄alkoxy-C₁₋₄alkoxy and R3 is methyl or methoxy; still more preferably wherein R2 is 3-methoxypropyloxy or 2-methoxyethyloxy and R3 is methoxy or methyl; yet more preferably wherein R2 is 2-methoxyethyloxy and R3 is methoxy; even more preferably wherein R2 is 3-methoxypropyloxy and R3 is methoxy or methyl; most preferably wherein R2 is 3-methoxypropyloxy and R3 is methoxy.

In another embodiment, preferred are the compounds of the formula (III), or salts thereof, wherein R2 is C₁₋₄alkoxy-C₁₋₄alkyl and R3 is C₁₋₈alkyl or C₁₋₈alkoxy; preferably wherein R2 is C₁₋₄alkoxy-C₁₋₄alkyl and R3 is C₁₋₈alkoxy; more preferably wherein R2 is C₁₋₄alkoxy-C₁₋₄alkyl and R3 is methoxy; even more preferably wherein R2 is 4-methoxybutyl and R3 is C₁₋₈alkoxy; most preferably wherein R2 is 4-methoxybutyl and R3 is methoxy.

In a further embodiment, preferred are the compounds of the formula (IV), or salts thereof, wherein R2 is C₁₋₄alkoxy-C₁₋₄alkoxy and R3 is C₁₋₈alkyl or C₁₋₈alkoxy; preferably wherein R2 is 3-methoxypropyloxy or 2-methoxyethyloxy and R3 is C₁₋₈alkyl or C₁₋₈alkoxy; more preferably wherein R2 is C₁₋₄alkoxy-C₁₋₄alkoxy and R3 is methyl or methoxy; still more preferably wherein R2 is 3-methoxypropyloxy or 2-methoxyethyloxy and R3 is methoxy or methyl; yet more preferably wherein R2 is 2-methoxyethyloxy and R3 is methoxy; even more preferably wherein R2 is 3-methoxypropyloxy and R3 is methoxy or methyl; most preferably wherein R2 is 3-methoxypropyloxy and R3 is methoxy.

As a result of the close relationship between the novel compounds in free form and in the form of their salts, hereinabove and hereinbelow any reference to the free compounds and their salts is to be understood as including also the corresponding salts and free compounds, respectively, as appropriate and expedient.

The compounds of the present invention may generally be prepared by those methods disclosed in U.S. Pat. No. 5,559,111, incorporated herein by reference in its entirety as if set forth in full herein.

The present invention further provides pharmaceutical compositions comprising a therapeutically effective amount of a pharmacologically active compound of the instant invention, alone or in combination with one or more pharmaceutically acceptable carriers.

The pharmaceutical compositions according to the present invention are those suitable for enteral, such as oral or rectal, transdermal and parenteral administration to mammals, including man, to inhibit renin activity, and for the treatment of conditions associated with renin activity. Such conditions include hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders.

Thus, the pharmacologically active compounds of the invention may be employed in the manufacture of pharmaceutical compositions comprising an effective amount thereof in conjunction or admixture with excipients or carriers suitable for either enteral or parenteral application. Preferred are tablets and gelatin capsules comprising the active ingredient together with:

a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol; for tablets also

c) binders, e.g., magnesium aluminum silicate, starch paste, gelatin, tragacanth, methylcellulose, sodium carboxymethylcellulose and or polyvinylpyrrolidone; if desired

d) disintegrants, e.g., starches, agar, alginic acid or its sodium salt, or effervescent mixtures; and/or

e) absorbants, colorants, flavors and sweeteners.

Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and pre-determined rate over a prolonged period of time, and means to secure the device to the skin.

Accordingly, the present invention provides pharmaceutical compositions as described above for the treatment of conditions mediated by renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders.

The pharmaceutical compositions may contain a therapeutically effective amount of a compound of the invention as defined above, either alone or in a combination with another therapeutic agent, e.g., each at an effective therapeutic dose as reported in the art. Such therapeutic agents include:

a) antidiabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; peroxisome proliferator-activated receptor (PPAR) ligands; protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose cotransporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; and DPPIV (dipeptidyl peptidase IV) inhibitors such as LAF237;

b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;

c) anti-obesity agents such as orlistat; and

d) anti-hypertensive agents, e.g., loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na-K-ATPase membrane pump such as digoxin; neutralendopeptidase (NEP) inhibitors; ACE/NEP inhibitors such as omapatrilat, sampatrilat and fasidotril; angiotensin II antagonists such as candesartan, eprosartan, irbesartan, losartan, telmisartan and valsartan, in particular valsartan; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors.

Other specific anti-diabetic compounds are described by Patel Mona in Expert Opin Investig Drugs, 2003, 12(4), 623-633, in the FIGS. 1 to 7, which are herein incorporated by reference. A compound of the present invention may be administered either simultaneously, before or after the other active ingredient, either separately by the same or different route of administration or together in the same pharmaceutical formulation.

The structure of the therapeutic agents identified by code numbers, generic or trade names may be taken from the actual edition of the standard compendium “The Merck Index” or from databases, e.g., Patents International (e.g. IMS World Publications). The corresponding content thereof is hereby incorporated by reference.

Accordingly, the present invention provides pharmaceutical compositions comprising a therapeutically effective amount of a compound of the invention in combination with a therapeutically effective amount of another therapeutic agent, preferably selected from anti-diabetics, hypolipidemic agents, anti-obesity agents or anti-hypertensive agents, most preferably from antidiabetics, anti-hypertensive agents or hypolipidemic agents as described above.

The present invention further relates to pharmaceutical compositions as described above for use as a medicament.

The present invention further relates to use of pharmaceutical compositions or combinations as described above for the preparation of a medicament for the treatment of conditions mediated by renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders.

Thus, the present invention also relates to a compound of formula (I) for use as a medicament, to the use of a compound of formula (I) for the preparation of a pharmaceutical composition for the prevention and/or treatment of conditions mediated by renin activity, and to a pharmaceutical composition for use in conditions mediated by renin activity comprising a compound of formula (I), or a pharmaceutically acceptable salt thereof, in association with a pharmaceutically acceptable diluent or carrier therefor.

The present invention further provides a method for the prevention and/or treatment of conditions mediated by renin activity, which comprises administering a therapeutically effective amount of a compound of the present invention.

A unit dosage for a mammal of about 50-70 kg may contain between about 1 mg and 1000 mg, advantageously between about 5-600 mg of the active ingredient. The therapeutically effective dosage of active compound is dependent on the species of warm-blooded animal (mammal), the body weight, age and individual condition, on the form of administration, and on the compound involved.

In accordance with the foregoing the present invention also provides a therapeutic combination, e.g., a kit, kit of parts, e.g., for use in any method as defined herein, comprising a compound of formula (I), or a pharmaceutically acceptable salt thereof, to be used concomitantly or in sequence with at least one pharmaceutical composition comprising at least another therapeutic agent, preferably selected from anti-diabetic agents, hypolipidemic agents, anti-obesity agents or anti-hypertensive agents. The kit may comprise instructions for its administration.

Similarly, the present invention provides a kit of parts comprising: (i) a pharmaceutical composition of the invention; and (ii) a pharmaceutical composition comprising a compound selected from an anti-diabetic, a hypolipidemic agent, an anti-obesity agent, an anti-hypertensive agent, or a pharmaceutically acceptable salt thereof, in the form of two separate units of the components (i) to (ii).

Likewise, the present invention provides a method as defined above comprising co-administration, e.g., concomitantly or in sequence, of a therapeutically effective amount of a compound of formula (I), or a pharmaceutically acceptable salt thereof, and a second drug substance, said second drug substance being an anti-diabetic, a hypolipidemic agent, an anti-obesity agent or an anti-hypertensive agent, e.g., as indicated above.

Preferably, a compound of the invention is administered to a mammal in need thereof.

Preferably, a compound of the invention is used for the treatment of a disease which responds to modulation of renin activity.

Preferably, the condition associated with renin activity is selected from hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders.

Finally, the present invention provides a method or use which comprises administering a compound of formula (I) in combination with a therapeutically effective amount of an anti-diabetic agent, a hypolipidemic agent, an anti-obesity agent or an anti-hypertensive agent.

Ultimately, the present invention provides a method or use which comprises administering a compound of formula (I) in the form of a pharmaceutical composition as described herein.

As used throughout the specification and in the claims, the term “treatment” embraces all the different forms or modes of treatment as known to those of the pertinent art and in particular includes preventive, curative, delay of onset and/or progression, and palliative treatment.

The above-cited properties are demonstrable in vitro and in vivo tests using advantageously mammals, e.g., mice, rats, rabbits, dogs, monkeys or isolated organs, tissues and preparations thereof. Said compounds can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and 10⁻¹⁰ molar concentrations. A therapeutically effective amount in vivo may range depending on the route of administration, between about 0.001 and 500 mg/kg, preferably between about 0.1 and 100 mg/kg.

As described above, the compounds of the present invention have enzyme-inhibiting properties. In particular, they inhibit the action of the natural enzyme renin. Renin passes from the kidneys into the blood where it effects the cleavage of angiotensinogen, releasing the decapeptide angiotensin I which is then cleaved in the lungs, the kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume which increase can be attributed to the action of angiotensin II. Inhibitors of the enzymatic activity of renin lead to a reduction in the formation of angiotensin I, and consequently a smaller amount of angiotensin II is produced. The reduced concentration of that active peptide hormone is the direct cause of the hypotensive effect of renin inhibitors.

The action of renin inhibitors may be demonstrated inter alia experimentally by means of in vitro tests, the reduction in the formation of angiotensin I being measured in various systems (human plasma, purified human renin together with synthetic or natural renin substrate).

Inter alia the following in vitro tests may be used:

An extract of human renin from the kidney (0.5 mGU [milli-Goldblaft units]/mL) is incubated for one h at 37° C. and pH 7.2 in 1M aqueous 2-N-(tris-hydroxymethylmethyl)-amino-ethanesulfonic acid buffer solution with 23 μg/mL of synthetic renin substrate, the tetradecapeptide H-Asp-Arg-Val-Tyr-Ile-His-ProPhe-His-Leu-Leu-Val-Tyr-Ser-OH. The amount of angiotensin I formed is determined by radioimmunoassay. Each of the inhibitors according to the invention is added to the incubation mixture at different concentrations. The IC₅₀ is defined as the concentration of a particular inhibitor that reduces the formation of angiotensin I by 50%.

Recombinant human renin (expressed in Chinese Hamster Ovary cells and purified using standard methods) at 4 nM concentration is incubated with test compound at various concentrations for 1 h at RT in 0.1 M Tris-HCl buffer, pH 7.4, containing 0.05 M NaCl, 0.5 mM EDTA and 0.05% CHAPS. Synthetic peptide substrate Arg-Glu(EDANS)-Ile-His-Pro-Phe-His-Leu-Val-Ile_His_Thr-Lys(DABCYL)-Arg9 is added to a final concentration of 2 μM and increase in fluorescence is recorded at an excitation wave-length of 340 nm and at an emission wave-length of 485 nm in a microplate spectro-fluorimeter. IC₅₀ values are calculated from percentage of inhibition of renin activity as a function of test compound concentration (Fluorescence Resonance Energy Transfer, FRET, assay). Recombinant human renin (expressed in Chinese Hamster Ovary cells and purified using standard methods) at 1 nM concentration is incubated with test compound at various concentrations for 1.5 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-His-Leu-Val-Ile-His-Asn-Lys-[DY-505-X5] is added to a final concentration of 5 μM. The enzyme reaction is stopped by adding 6 μL of 1.0% TFA. The product of the reaction is separated by HPLC and quantified by spectrophotometric measurement at 505 nM wave-length. IC₅₀ values are calculated from percentage of inhibition of renin activity as a function of test compound concentration.

Recombinant human renin (expressed in Chinese Hamster Ovary cells and purified using standard methods) at 3.3 nM concentration, 125I-NVP-AJI891-NX-1 (0.27 μCi/mL) and streptavidin-SPA (0.67 mg/mL) beads are incubated with test compound at various concentrations for 2.0 h at RT in 0.1 M Tris/HCl pH 7.4 containing 0.5M NaCl and 0.5% (w/v) Brij35. At the end of the incubation time, the plates are centrifuged (55 g, 60 seconds) and counted in a Wallac MicroBeta reader. IC₅₀ values are calculated from percentage of displacement of radioligand binding to renin as a function of test compound concentration.

In animals deficient in salt, renin inhibitors bring about a reduction in blood pressure. Human renin may differ from the renin of other species. In order to test inhibitors of human renin, primates, e.g., marmosets (Callithrix jacchus) may be used, because human renin and primate renin are substantially homologous in the enzymatically active region. Inter alia the following in vivo tests may be used:

The test compounds are tested on normotensive marmosets of both sexes having a body weight of approximately 350 g that are conscious, allowed to move freely and in their normal cages. The blood pressure and heart rate are measured via a catheter in the descending aorta and recorded radiometrically. The endogenous release of renin is stimulated by the combination of a 1-week low-salt diet and a single intramuscular injection of furosemide (5-(aminosulfonyl)-4-chloro-2-[(2-furanylmethyl)amino]benzoic acid) (5 mg/kg). 16 h after the injection of furosemide the test compounds are administered either directly into the femoral artery using an injection cannula or, in the form of a suspension or solution, via an oesophageal tube into the stomach, and their action on the blood pressure and heart rate are evaluated. In the in vivo test described, the compounds of the present invention have hypotensive action at doses of from approximately 0.003 to approximately 1 mg/kg i.v. and at doses of from approximately 0.3 to approximately 100 mg/kg p.o.

Alternatively, renin inhibitors may be tested on male normotensive marmosets weighing 250 to 500 g that are conscious, allowed to move freely and in their normal cages. The blood pressure, and heart rate are measured via a catheter placed in the descending aorta and recorded radiometrically. Electrocardiogram are obtained by placing electrodes of transmitter in lead II. The endogenous release of renin is stimulated by two intramuscular injection of furosemide (5-(aminosulfonyl)-4-chloro-2-[(2-furanylmethyl)amino]benzoic acid) (10 mg/kg) 43 and 19 hours prior compound application. Test compounds are administered either directly into the femoral artery using an injection cannula or, in the form of a suspension or solution, via an oesophageal tube into the stomach, and their action on the blood pressure, heart rate and ECG are evaluated. In the in vivo test described, compounds of the present invention have hypotensive action at doses of from approximately 0.003 to approximately 0.3 mg/kg i.v. and at doses of from approximately 0.31 to approximately 30 mg/kg p.o.

The compounds of the present invention also have the property of regulating, especially reducing, intra-ocular pressure.

The extent of the reduction in intra-ocular pressure after administration of a pharmaceutical active ingredient of formula (I) according to the present invention can be determined, for example, in animals, for example rabbits or monkeys. Two typical experimental procedures that illustrate the present invention, but are not limited to in any way, are described hereinafter.

The in vivo test on a rabbit of the “Fauve de Bourgogne” type to determine the intra-ocular-pressure-reducing activity of topically applied compositions can be designed, for example, as follows: The intra-ocular pressure (IOP) is measured using an aplanation tonometer both before the experiment and at regular intervals of time. After a local anaesthetic has been administered, the suitably formulated test compound is applied topically in a precisely defined concentration (e.g. 0.000001-5% by weight) to one eye of the animal in question. The contralateral eye is treated, for example, with physiological saline. The measured values thus obtained are evaluated statistically.

The in vivo tests on monkeys of the species Macaca Fascicularis to determine the intra-ocular-pressure-reducing activity of topically applied compositions can be carried out, e.g., as follows: The suitably formulated test compound is applied in a precisely defined concentration (e.g. 0.000001-5% by weight) to one eye of each monkey. The other eye of the monkey is treated correspondingly, for example with physiological saline. Before the start of the test the animals are anaesthetised with intramuscular injections of, for example, ketamine. At regular intervals of time, the intra-ocular pressure (IOP) is measured. The test is carried out and evaluated in accordance with the rules of “good laboratory practice” (GLP).

It has been found that the new compounds, beside being potent renin inhibitors, also show improved biological properties, for example, efficacy and/or bioavailability, over previously disclosed renin inhibitors, such as the compounds of U.S. Pat. No. 5,559,111.n particular aliskiren (Example 83), which is marketed under the name of Tekturna.

Pharmacokinetic profiles are investigated in male Sprague-Dawley rats implanted with jugular vein catheters. Compounds are administered orally in 0.5% aqueous methylcellulose solution or intravenously in N-methylpyrrolidinone-PEG200 (10:90, v/v). Typical doses are 6 mg/kg p.o. and 2 mg/kg i.v., respectively. Blood samples are serially taken through venous catheters into heparinized tubes at various time points until 32 h post dose and plasma is separated by centrifugation.

Plasma concentrations of the compounds described in this invention are measured by liquid chromatography-tandem mass spectrometry after extraction with acetonitrile.

Pharmacokinetic parameters are calculated by using a non-compartmental method.

The following Examples are intended to illustrate the invention and are not to be construed as being limitations thereon. If not mentioned otherwise, all evaporations are performed under reduced pressure, preferably between about 10 and 100 mmHg (=20-133 mbar). The structure of final products, intermediates and starting materials is confirmed by standard analytical methods, e.g., microanalysis, melting point (m.p.) and spectroscopic characteristics, e.g., MS, LC/MS, IR, NMR. In general, abbreviations used are those conventional in the art.

### Abbreviations:

AcOH acetic acid
DIBAL-H diisobutylaluminium hydride
4-DMAP 4-dimethylamino-pyridine
DMF dimethylformamide
DMSO dimethylsulfoxide
EtOAc ethyl acetate
NEt₃ triethylamine
EtOH ethanol
Flow flow rate
h hour(s)
HBTU O-(Benzotriazol-1-yloxy)-N,N,N′,N′-tetramethyluronium-hexafluorophosphate
HMPA hexamethylphosphoroamide
HPLC High Performance Liquid Chromatography
L liter(s)
LC-MS Liquid Chromatography/Mass Spectrometry
Me methyl
MeOH methanol
min minute(s)
mL milliliter
MS Mass Spectrometry
Pd/C palladium on charcoal
PyBOP (Benzotriazol-1-yloxy)-tripyrrolidinophosphonium-hexafluorophosphate
RT room temperature
TBAF tetra-butylammonium fluoride
TBDMS-Cl tert-butyldimethylsilyl chloride
TBDMS tert-butyldimethylsilyl
TFA trifluoroacetic acid
THF tetrahydrofuran
RP reversed phase
TLC Thin Layer Chromatography
tᵣ retention time

Temperatures are measured in degrees Celsius. Unless otherwise indicated, the reactions were performed at RT.

TLC conditions: R_{f} values for TLC are measured on 5×10 cm TLC plates, silica gel F₂₅₄, Merck, Darmstadt, Germany.

The general procedures to produce compounds of formula I are exemplified in Synthetic Routes (I) to (III) and as described in more detail in the Examples.

### General Synthetic Route (I):

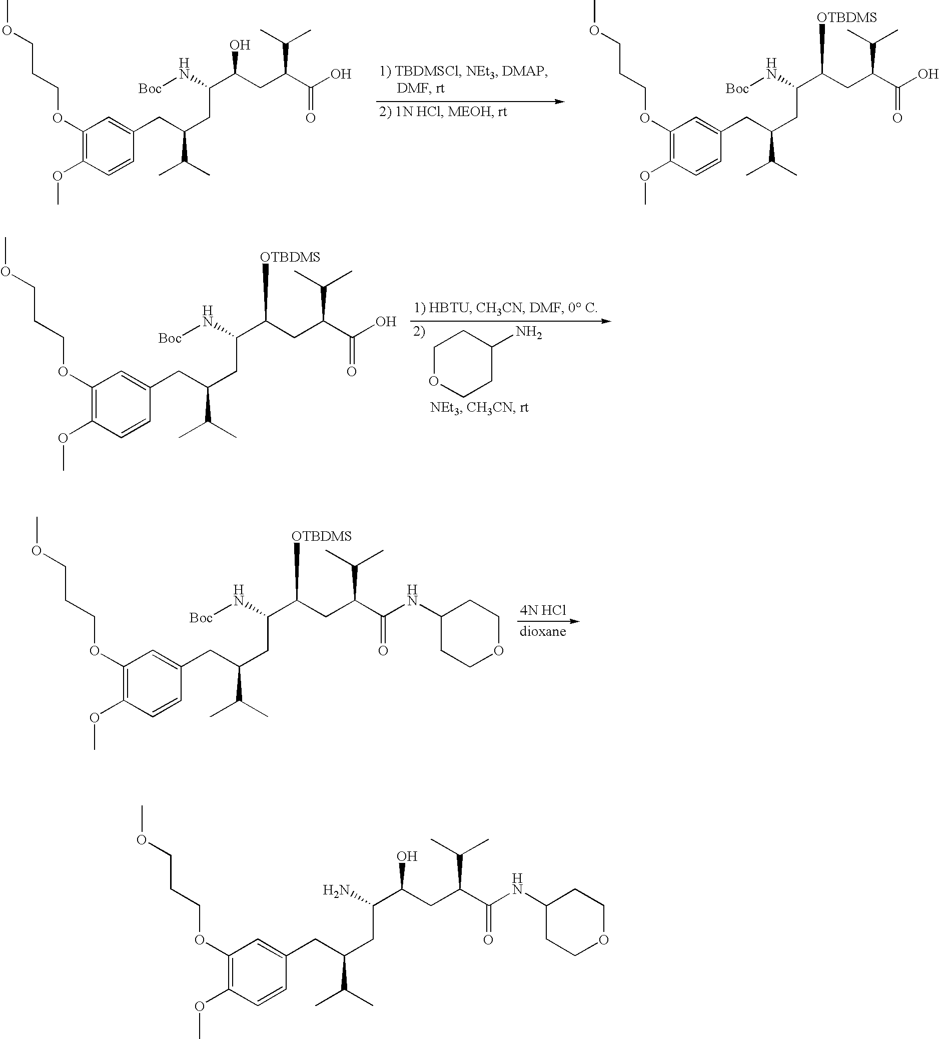

### General Synthetic Route (II):

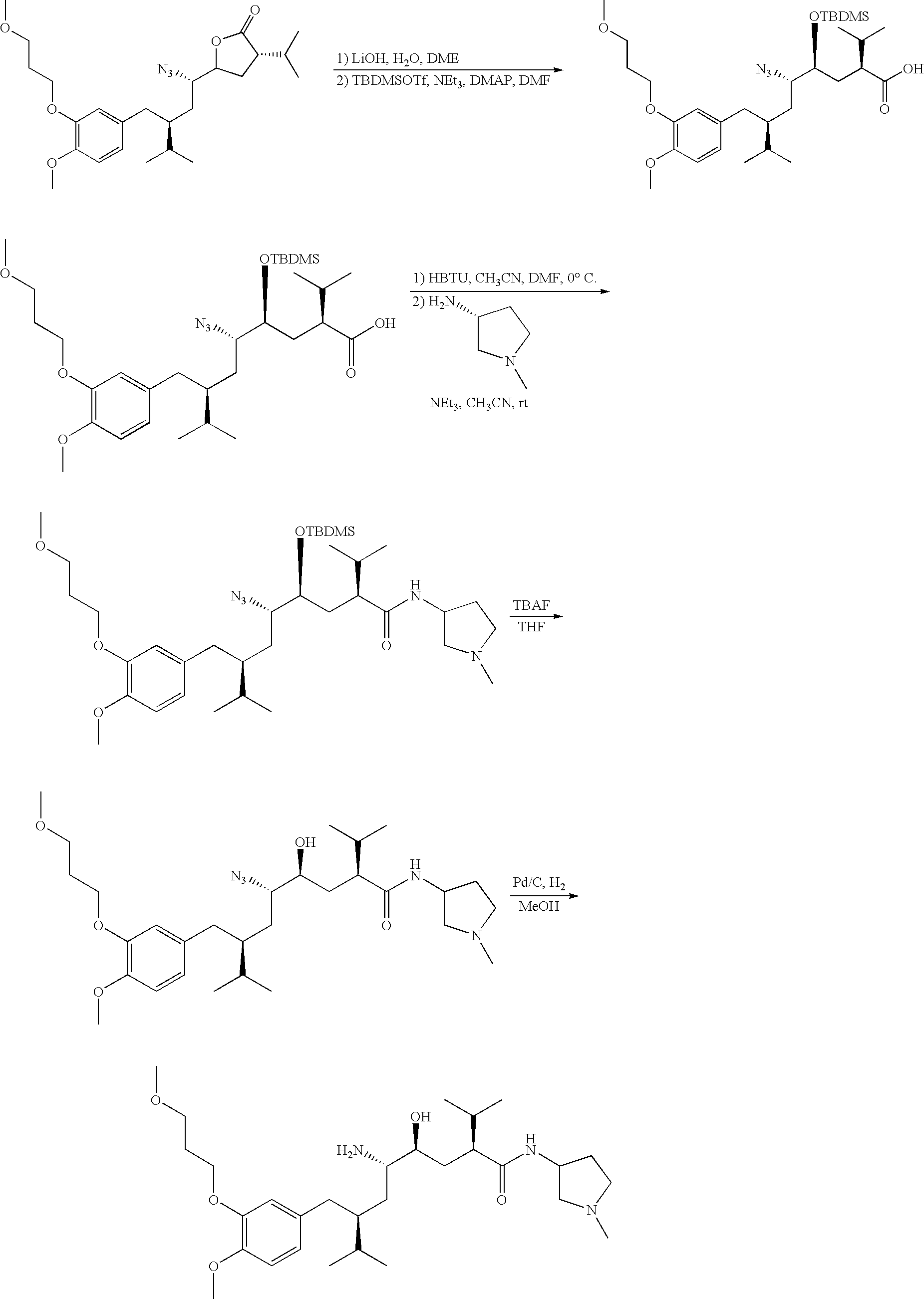

### General Synthetic Route (III):

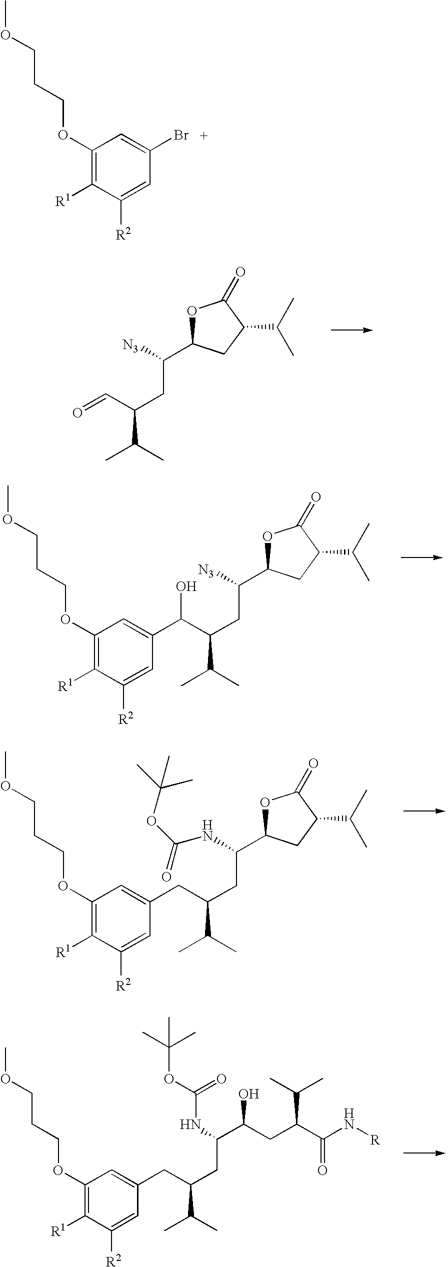

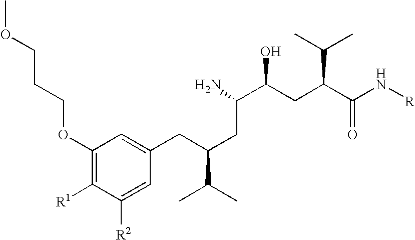

### Example 1

### General Synthetic Route 1

### (2S,4S,5S,7S)-5-tert-Butoxycarbonylamino-4-(tert-butyl-dimethyl-silanyloxy)-2-isopropyl-7-[4-methoxy-3-(3-methoxy-propoxy)-benzyl]-8-methyl-nonanoic acid

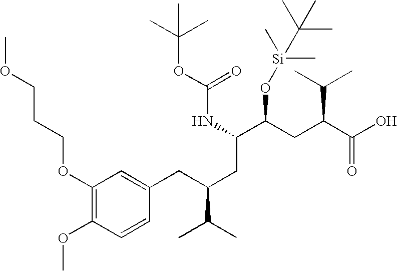

To a solution of (2S,4S,5S,7S)-5-tert-butoxycarbonylamino-4-hydroxy-2-isopropyl-7-[4-methoxy-3-(3-methoxy-propoxy)-benzyl]-8-methyl-nonanoic acid [853273-50-4] (9.53 g, 17.2 mmol, 1.0 eq; prepared according to EP0678503A1) and TBDMS-Cl (10.3 g, 68.7 mmol, 4.0 eq) in DMF (100 ml) are added NEt₃ (7.2 ml, 51.6 mmol, 3.0 eq) followed by 4-DMAP (640 mg, 5.2 mmol, 0.3 eq) at RT. The reaction mixture is stirred at rt for 16 h, followed by addition of water. Extraction with EtOAc, drying (Na₂SO₄) and evaporation of the solvent affords the crude product. Flash column chromatography (n-hexane/EtOAc 5:1) yields the double TBDMS-protected product as a colorless oil.

A portion thereof (904 mg, 1.24 mmol, 1.0 eq) is dissolved in MeOH (20 ml), and 1M HCl (2 ml, 2 mmol, 1.6 eq) is added. The mixture is stirred at RT for 10 min before 1M NaOH (2 ml) followed by water and a 10% citric acid solution are added for workup. Extraction with EtOAc, drying (Na₂SO₄) of the combined organic extracts and evaporation of the solvent give the crude product which is purified by flash column chromatography (CH₂Cl₂/MeOH 9:1) to afford the desired product as a colorless oil. t_{R} (HPLC, C8 column, 5-95% CH₃CN/H₂O/6.5 min, 95% CH₃CN/H₂O/1 min, flow: 0.5 ml/min): 7.63 min. MS (LC-MS): 691.3 [M+Na]⁺.

### [(1S,2S,4S)-2-(tert-Butyl-dimethyl-silanyloxy)-1-{(S)-2-[4-methoxy-3-(3-methoxy-propoxy)-benzyl]-3-methyl-butyl}-5-methyl-4-(tetrahydro-pyran-4-ylcarbamoyl)-hexyl]-carbamic acid tert-butyl ester

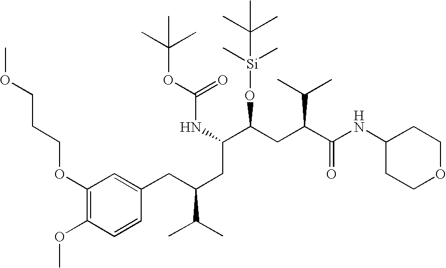

To a solution of (2S,4S,5S,7S)-5-tert-butoxycarbonylamino-4-(tert-butyl-dimethyl-silanyloxy)-2-isopropyl-7-[4-methoxy-3-(3-methoxy-propoxy)-benzyl]-8-methyl-nonanoic acid (548 mg, 0.82 mmol, 1.0 eq) in CH₃CN (6 ml) and DMF (1 ml) is added HBTU (381 mg, 0.98 mmol, 1.2 eq) at 0° C. After 5 min, a solution of tetrahydro-pyran-4-ylamine (83 mg, 0.82 mmol, 1.0 eq) and NEt₃ (1.2 ml, 8.2 mmol, 10 eq) in CH₃CN (6 ml) is added and the reaction mixture is stirred at RT for 15 min. For workup, EtOAc is added and the organic layer is washed with 1N HCl a saturated solution of NaHCO₃ and brine. Drying (Na₂SO₄) of the organic phase and evaporation of the solvent affords the crude product which is purified by flash column chromatography (n-hexane/EtOAc 1:1) to afford the desired product as a colorless foam. R_{f} (n-hexane/EtOAc 1:1): 0.22. MS (LC-MS): 751.4 [M+H]⁺.

### (2S,4S,5S,7S)-5-Amino-4-hydroxy-2-isopropyl-7-[4-methoxy-3-(3-methoxy-propoxy)-benzyl]-8-methyl-nonanoic acid (tetrahydro-pyran-4-yl)-amide

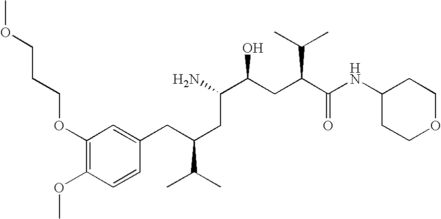

To a solution of [(1S,2S,4S)-2-(tert-butyl-dimethyl-silanyloxy)-1-{(S)-2-[4-methoxy-3-(3-methoxy-propoxy)-benzyl]-3-methyl-butyl}-5-methyl-4-(tetrahydro-pyran-4-ylcarbamoyl)-hexyl]-carbamic acid tert-butyl ester (938 mg, 1.25 mmol, 1.0 eq) in dioxane (8 mL) is added 4N HCl in dioxane (2 mL, 8 mmol). The resulting solution is stirred at RT for 1 h, followed by careful addition of a saturated solution of NaHCO₃. The mixture is extracted with CH₂Cl₂, the combined extracts are dried (Na₂SO₄) and the solvent is evaporated. Flash column chromatography (CH₂Cl₂/MeOH 4:1) affords the product as a colorless oil. t_{R} (HPLC, C8 column, 5-95% CH₃CN/H₂O/6.5 min, 95% CH₃CN/H₂O/1 min, flow: 0.5 ml/min): 4.22 min. MS (LC-MS): 537.3 [M+H]⁺.

### Example 2

### General Synthetic Route 2

### (2S,4S,5S,7S)-5-Azido-4-(tert-butyl-dimethyl-silanyloxy)-2-isopropyl-7-[4-methoxy-3-(3-methoxy-propoxy)-benzyl]-8-methyl-nonanoic acid

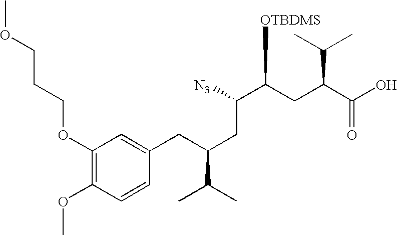

To a solution of (3S,5S)-5-{(1S,3S)-1-azido-3-[4-methoxy-3-(3-methoxy-propoxy)-benzyl]-4-methyl-pentyl}-3-isopropyl-dihydro-furan-2-one [324763-46-4] (20.0 g, 43.3. mmol) in DME (400 ml) and H₂O (200 ml) is added LiOH.H₂O (2.18 g, 52.0 mmol). After stirring at RT for 2 h, the solvent is co-evaporated with toluene and the resulting solid is dried under high vacuum. This residue is dissolved in DMF (160 ml) followed by addition of NEt₃ (32 ml, 227.6 mmol), TBDMSOTf (41.8 ml, 182.1 mmol) and 4-DMAP (556 mg, 4.6 mmol) and the mixture is stirred at RT for 16 h. For workup, EtOAc is added and the mixture is quenched by addition of a saturated solution of NaHCO₃. The organic phase is separated and the aqueous phase is extracted with EtOAc. Evaporation of the solvent of the combined organic extracts affords bis-TBDMS protected product. Acidification of the basic aqueous layer with 1N HCl followed by extraction with EtOAc and evaporation of the solvent yields the corresponding mono-silylated free acid. Both isolated products are combined and subjected to flash column chromatography (n-hexane/EtOAc gradient 4:1 to 1:1) to give the title compound as a viscous oil. t_{R} (HPLC, C8 column, 20-95% CH₃CN/H₂O/3.5 min, 95% CH₃CN/1 min, flow: 0.8 ml/min): 3.93 min. MS (LC-MS): 616.0 [M+Na]⁺.

### (2S,4S,5S,7S)-5-Azido-4-(tert-butyl-dimethyl-silanyloxy)-2-isopropyl-7-[4-methoxy-3-(3-methoxy-propoxy)-benzyl]-8-methyl-nonanoic acid ((R)-1-methyl-pyrrolidin-3-yl)-amide

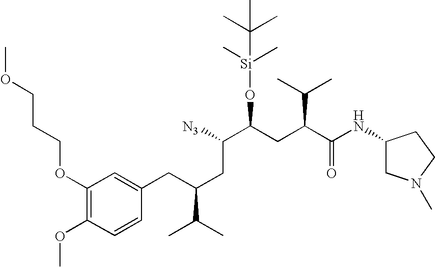

To a solution of (2S,4S,5S,7S)-5-azido-4-(tert-butyl-dimethyl-silanyloxy)-2-isopropyl-7-[4-methoxy-3-(3-methoxy-propoxy)-benzyl]-8-methyl-nonanoic acid (1.50 g, 2.5 mmol) in CH₃CN (50 mL) is added HBTU (1.2 g, 3.0 mmol) at 0° C. After 5 min, (R)-1-methyl-pyrrolidin-3-ylamine hydrochloride salt (875 mg, 5.1 mmol) and NEt₃ (3.9 mL, 27.8 mmol) are added. The solution is allowed to warm to RT and stirring is continued for another 1.5 h. For workup, EtOAc is added and the organic phase is washed with 1N HCl a saturated solution of NaHCO₃ and brine, followed by drying (Na₂SO₄). The solvent is evaporated and the crude product is purified by flash column chromatography (CH₂Cl₂/MeOH 95:5) to give the title compound as a colorless foam. t_{R} (HPLC, C18 column, 10-100% CH₃CN/H₂O/5 min, 100% CH₃CN/3 min, 100-10% CH₃CN/H₂O/3 min, flow: 1.5 ml/min): 6.8 min. MS (LC-MS): 676.1 [M+H]⁺.

### (2S,4S,5S,7S)-5-Azido-4-hydroxy-2-isopropyl-7-[4-methoxy-3-(3-methoxy-propoxy)-benzyl]-8-methyl-nonanoic acid ((R)-1-methyl-pyrrolidin-3-yl)-amide

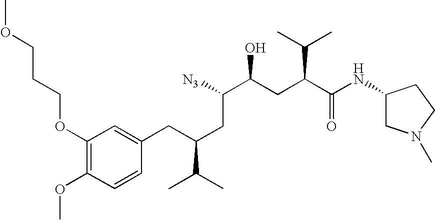

To a solution of (2S,4S,5S,7S)-5-azido-4-(tert-butyl-dimethyl-silanyloxy)-2-isopropyl-7-[4-methoxy-3-(3-methoxy-propoxy)-benzyl]-8-methyl-nonanoic acid ((R)-1-methyl-pyrrolidin-3-yl)-amide (1.37 g, 2.0 mmol) in THF (15 mL) is added TBAF·3H₂O (1.6 g, 5.1. mmol), followed by stirring at RT overnight. For workup, water is added and the mixture is extracted with CH₂Cl₂. The combined organic extracts are dried (Na₂SO₄) and the solvent is evaporated. Flash column chromatography (CH₂Cl₂/MeOH 9:1) yields the desired product as a yellowish oil. t_{R} (HPLC, C18 column, 10-100% CH₃CN/H₂O/5 min, 100% CH₃CN/3 min, 100-10% CH₃CN/H₂O/3 min, flow: 1.5 ml/min): 5.08 min. MS (LC-MS): 562.1 [M+H]⁺.

### (2S,4S,5S,7S)-5-Amino-4-hydroxy-2-isopropyl-7-[4-methoxy-3-(3-methoxy-propoxy)-benzyl]-8-methyl-nonanoic acid ((R)-1-methyl-pyrrolidin-3-yl)-amide

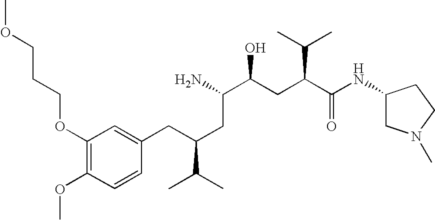

To a solution of (2S,4S,5S,7S)-5-azido-4-hydroxy-2-isopropyl-7-[4-methoxy-3-(3-methoxy-propoxy)-benzyl]-8-methyl-nonanoic acid ((R)-1-methyl-pyrrolidin-3-yl)-amide (400 mg, 0.7 mmol) in MeOH (20 mL) is added Pd/C 10%. The suspension is stirred under an atmosphere of hydrogen until completion of the reaction. The catalyst is filtered-off over Celite® and washed with MeOH. Evaporation of the solvent gives the crude product which is purified by preparative HPLC to afford the desired product as a colorless foam. t_{R} (HPLC, C18 column, 10-100% CH₃CN/H₂O/5 min, 100% CH₃CN/3 min, 100-10% CH₃CN/H₂O/3 min, flow: 1.5 ml/min): 4.30 min. MS (LC-MS): 536.1 [M+H]⁺.

The starting material is prepared as follows:

### a) 1-Methyl-3-(R)-aminopyrrolidine

1-Methyl-3-(R)—BOC-aminopyrrolidine (1.5 g, 7.5 mmol) is dissolved in dioxane (10 mL), and 4N HCl in dioxane (7.5 mL, 30 mmol) is added followed by stirring at RT for 4 h. Lyophilization affords the title compound as its hydrochloride salt as a colorless foam.

### b) 1-Methyl-3-(R)—BOC-aminopyrrolidine

To a solution of 3-(R)—BOC-aminopyrrolidine (2.0 g, 10.7 mmol) in MeOH (40 mL) is added a 37% formaldehyde solution in water (2.4 mL) at RT, followed by addition of NaBH₄ (1.22 g, 32.2. mmol; exothermic reaction) at 0° C. The mixture is allowed to warm to RT and stirred for another 15 h. For workup, water is added and the mixture is extracted with CH₂Cl₂, the combined extracts are dried (Na₂SO₄) and the solvent is evaporated to give the title compound as a yellow powder.

### Example 3

### (2S,4S,5S,7S)-5-Amino-4-hydroxy-2-isopropyl-7-[4-methoxy-3-(3-methoxy-propoxy)-benzyl]-8-methyl-nonanoic acid ((S)-1-methyl-pyrrolidin-3-yl)-amide

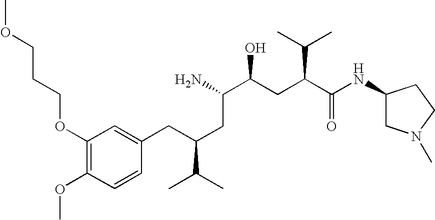

The title compound is prepared in accordance to Example 2. t_{R} (HPLC, C18 column, 10-100% CH₃CN/H₂O/5 min, 100% CH₃CN/3 min, 100-10% CH₃CN/H₂O/3 min, flow; 1.5 ml/min): 4.20 min. MS (LC-MS): 536.1 [M+H]⁺.

The corresponding amine (3-(S)-aminopyrrolidine) is prepared from 3-(S)—BOC-aminopyrrolidine in a similar fashion as described for (3-(R)-aminopyrrolidine) in Example 2a and 2b).

### Example 4

### (2S,4S,5S,7S)-5-Amino-4-hydroxy-2-isopropyl-7-[4-methoxy-3-(3-methoxy-propoxy)-benzyl]-8-methyl-nonanoic acid (1-aza-bicyclo[2.2.2]oct-3-yl)-amide

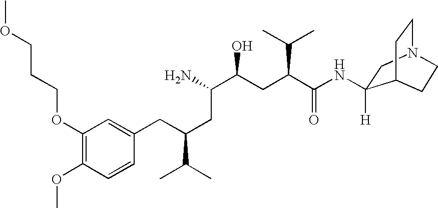

The title compound is prepared as follows:

A solution of ((1S,2S,4S)-4-(1-aza-bicyclo[2.2.2]oct-3-ylcarbamoyl)-2-hydroxy-1-{(S)-2-[4-methoxy-3-(3-methoxy-propoxy)-benzyl]-3-methyl-butyl}-5-methyl-hexyl)-carbamic acid tert-butyl ester (378 mg, 0.57 mmol) in 4N HCl/dioxane (7 mL) is stirred at RT for 1 h. The free base product is isolated by preparative HPLC, dissolved in dioxane (8 mL) and transformed into the corresponding bis-hydrochloride salt by treatment with 4N HCl/dioxane (116 μL, 0.46 mmol) followed by freeze-drying. t_{R} (HPLC, C18 column, 10-100% CH₃CN/H₂O/5 min, 100% CH₃CN/3 min, 100-10% CH₃CN/H₂O/3 min, flow: 1.5 ml/min): 4.35 min. MS (LC-MS): 562.1 [M+H]⁺.

The starting material is prepared as follows:

### ((1S,2S,4S)-4-(1-Aza-bicyclo[2.2.2]oct-3-ylcarbamoyl)-2-hydroxy-1-{(S)-2-[4-methoxy-3-(3-methoxy-propoxy)-benzyl]-3-methyl-butyl}-5-methyl-hexyl)-carbamic acid tert-butyl ester

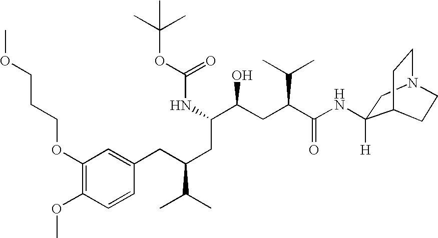

A solution of ((1S,2S,4S)-4-(1-aza-bicyclo[2.2.2]oct-3-ylcarbamoyl)-2-(tert-butyl-dimethyl-silanyloxy)-1-{(S)-2-[4-methoxy-3-(3-methoxy-propoxy)-benzyl]-3-methyl-butyl}-5-methyl-hexyl)-carbamic acid tert-butyl ester (860 mg, 1.11 mmol), prepared according to Example 1 by peptide coupling of (2S,4S,5S,7S)-5-tert-butoxycarbonylamino-4-(tert-butyl-dimethyl-silanyloxy)-2-isopropyl-7-[4-methoxy-3-(3-methoxy-propoxy)-benzyl]-8-methyl-nonanoic acid and (R)-(1-aza-bicyclo[2.2.2]oct-3-yl)amine), and TBAF (1.02 g, 3.24 mmol) in THF (10 mL), is stirred at RT for 3 h. In order to complete the reaction, more TBAF (680 mg, 2.2 mmol) is added and the reaction is stirred for another 20 h. Water is added and the mixture is extracted with EtOAc. The combined organic extracts are dried (Na₂SO₄), the solvent is evaporated and the desired product is isolated by flash column chromatography (CH₂Cl₂/MeOH 9:1) as a colorless foam. t_{R} (HPLC, C18 column, 10-100% CH₃CN/H₂O/5 min, 100% CH₃CN/3 min, 100-10% CH₃CN/H₂O/3 min, flow: 1.5 ml/min): 5.17 min. MS (LC-MS): 662.1 [M+H]⁺.

### Example 5

### (2S,4S,5S,7S)-5-Amino-4-hydroxy-2-isopropyl-7-[4-methoxy-3-(3-methoxy-propoxy)-benzyl]-8-methyl-nonanoic acid (1-methyl-piperidin-4-yl)-amide

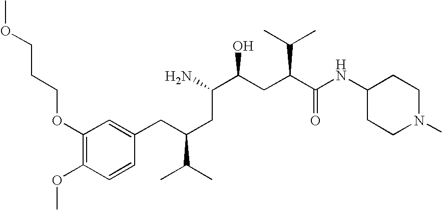

The title compound is prepared in accordance to Example 1. t_{R} (HPLC, C18 column, 10-100% CH₃CN/H₂O/5 min, 100% CH₃CN/3 min, 100-10% CH₃CN/H₂O/3 min, flow: 1.5 ml/min): 4.15 min. MS (LC-MS): 551.1 [M+H]⁺.

### Example 6

### (2S,4S,5S,7S)-5-Amino-4-hydroxy-2-isopropyl-7-[4-methoxy-3-(3-methoxy-propoxy)-benzyl]-8-methyl-nonanoic acid (1-benzyl-piperidin-4-yl)-amide

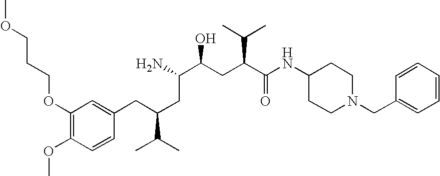

The title compound is prepared in accordance to Example 1. t_{R} (HPLC, C18 column, 10-100% CH₃CN/H₂O/5 min, 100% CH₃CN/3 min, 100-10% CH₃CN/H₂O/3 min, flow: 1.5 ml/min): 4.45 min. MS (LC-MS): 626.1 [M]⁺.

### Example 7

### 4-{(2S,4S,5S,7S)-5-Amino-4-hydroxy-2-isopropyl-7-[4-methoxy-3-(3-methoxy-propoxy)-benzyl]-8-methyl-nonanoylamino}-piperidine-1-carboxylic acid tert-butyl ester

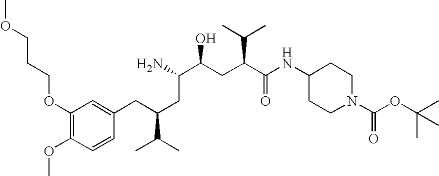

The title compound is prepared in accordance to Example 2. t_{R} (HPLC, C8 column, 5-95% CH₃CN/H₂O/6.5 min, 95% CH₃CN/H₂O/1 min, flow: 0.5 ml/min): 5.12 min. MS (LC-MS): 636.2 [M+H]⁺.

### Example 8

### (2S,4S,5S,7S)-5-Amino-4-hydroxy-2-isopropyl-7-[4-methoxy-3-(3-methoxy-propoxy)-benzyl]-8-methyl-nonanoic acid ((S)-2-oxo-tetrahydro-furan-3-yl)-amide

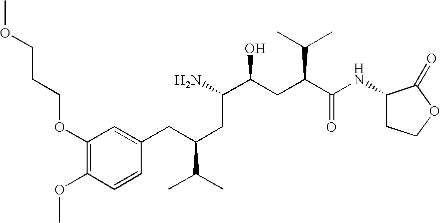

The title compound is prepared in accordance to Example 2. t_{R} (HPLC, C18 column, 10-100% CH₃CN/H₂O/5 min, 100% CH₃CN/3 min, 100-10% CH₃CN/H₂O/3 min, flow: 1.5 ml/min): 4.65 min. MS (LC-MS): 538.0 [M+H]⁺.

### Example 9

### (2S,4S,5S,7S)-5-Amino-4-hydroxy-2-isopropyl-7-[4-methoxy-3-(3-methoxy-propoxy)-benzyl]-8-methyl-nonanoic acid (3-methyl-oxetan-3-ylmethyl)-amide

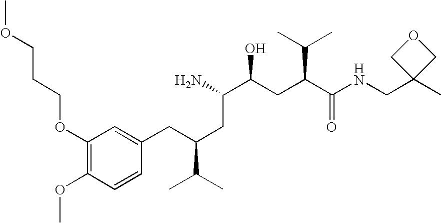

A solution of {(1S,2S,4S)-2-hydroxy-1-{(S)-2-[4-methoxy-3-(3-methoxy-propoxy)-benzyl]-3-methyl-butyl}-5-methyl-4-[(3-methyl-oxetan-3-ylmethyl)-carbamoyl]-hexyl}-carbamic acid tert-butyl ester (852 mg, 1.34 mmol) in 1,2-dichloroethane (2 mL) is treated with anhydrous ZnBr₂ (663 mg, 2.94 mmol), and the resulting suspension is heated at 50° C. overnight. The reaction mixture is poured into a mixture of water (10 mL) and saturated sodium bicarbonate solution (5 mL) and extracted with EtOAc. The combined organic phases are washed with water, dried (Na₂SO₄) and the solvent is evaporated. Purification of the crude product by preparative HPLC affords the desired product. t_{R} (HPLC, C8 column, 5-95% CH₃CN/H₂O/6.5 min, 95% CH₃CN/H₂O/1 min, flow: 0.5 ml/min): 4.51 min. MS (LC-MS): 537.1 [M+H]⁺.

The starting material is prepared as follows:

### {(1S,2S,4S)-2-Hydroxy-1-{(S)-2-[4-methoxy-3-(3-methoxy-propoxy)-benzyl]-3-methyl-butyl}-5-methyl-4-[(3-methyl-oxetan-3-ylmethyl)-carbamoyl]-hexyl}-carbamic acid tert-butyl ester

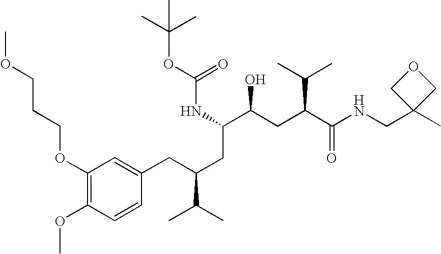

TBAF (950 mg, 3.01 mmol) is added to a solution of {(1S,2S,4S)-2-(tert-butyl-dimethyl-silanyloxy)-1-{(S)-2-[4-methoxy-3-(3-methoxy-propoxy)-benzyl]-3-methyl-butyl}-5-methyl-4-[(3-methyl-oxetan-3-ylmethyl)-carbamoyl]-hexyl}-carbamic acid tert-butyl ester (1.507 g, 2.01 mmol, prepared according to Example 1 by peptide coupling of (2S,4S,5S,7S)-5-tert-butoxycarbonylamino-4-(tert-butyl-dimethyl-silanyloxy)-2-isopropyl-7-[4-methoxy-3-(3-methoxy-propoxy)-benzyl]-8-methyl-nonanoic acid and C-(3-methyl-oxetan-3-yl)-methylamine), in THF (10 mL) at RT. After stirring overnight, water is added and the mixture is extracted with EtOAc. Drying of the combined extracts (Na₂SO₄) and evaporation of the solvent affords the crude product which is purified by flash column chromatography (hexane/ethyl acetate 1:1) to afford the desired product R_{f} (hexane/EtOAc 1:1): 0.16. MS (LC-MS): 637.1 [M+H]⁺.

The amine for peptide coupling is prepared as follows:

### a) C-(3-Methyl-oxetan-3-yl)-methylamine

To a solution of toluene-4-sulfonic acid 3-methyl-oxetan-3-ylmethyl ester (4.6 g, 18 mmol) in DMSO (15 mL) is added NaN₃ (3.5 g, 54 mmol) at RT and the resulting suspension is stirred overnight. Water is added and the mixture is extracted with diethyl ether. The organic solution containing the desired product 3-azidomethyl-3-methyl-oxetane is concentrated to half of its original volume and directly used without further purification. PPh₃ (4.6 g, 18 mmol) is slowly added to a well-stirred solution of the above-mentioned etheral solution of 3-azidomethyl-3-methyl-oxetane and the reaction mixture is stirred at RT overnight. Water (0.35 mL) is added and the mixture is heated under reflux for 2 h before the solvent is evaporated. Purification of the crude product by flash column chromatography (CH₂Cl₂ then CH₂Cl₂/CH₃OH 4:1) affords the title compound.

### b) Toluene-4-sulfonic acid 3-methyl-oxetan-3-ylmethyl ester

To a solution of (3-methyl-oxetan-3-yl)-methanol (3 mL, 30 mmol) and NEt₃ (4.7 mL, 33 mmol) in CH₂Cl₂ (30 mL) is added toluene-4-sulfonyl chloride (6.4 g, 33 mmol) at 0° C. The reaction is allowed to warm to RT overnight. For workup, a saturated solution of NaHCO₃ is added and the mixture is extracted with EtOAc. The crude product is purified by flash column chromatography (n-hexane/EtOAc 1:1) to give the title compound.

### Example 10

### (2S,4S,5S,7S)-5-Amino-4-hydroxy-2-isopropyl-7-[4-methoxy-3-(3-methoxy-propoxy)-benzyl]-8-methyl-nonanoic acid [(S)-1-(tetrahydro-furan-2-yl)methyl]-amide

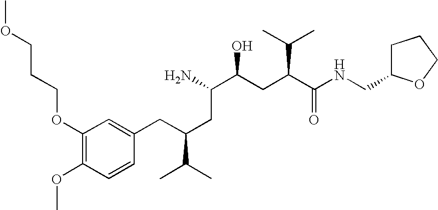

The title compound is prepared in accordance to Example 1. t_{R} (HPLC, C18 column, 10-100% CH₃CN/H₂O/5 min, 100% CH₃CN/3 min, 100-10% CH₃CN/H₂O/3 min, flow: 1.5 ml/min): 4.74 min. MS (LC-MS): 537.2 [M+H]⁺.

### Example 11

### (2S,4S,5S,7S)-5-Amino-4-hydroxy-2-isopropyl-7-[4-methoxy-3-(3-methoxy-propoxy)-benzyl]-8-methyl-nonanoic acid [(R)-1-(tetrahydro-furan-2-yl)methyl]-amide

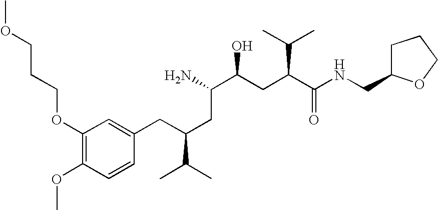

The title compound is prepared in accordance to Example 1. t_{R} (HPLC, C18 column, 10-100% CH₃CN/H₂O/5 min, 100% CH₃CN/3 min, 100-10% CH₃CN/H₂O/3 min, flow: 1.5 ml/min): 4.71 min. MS (LC-MS): 537.1 [M+H]⁺.

### Example 12

### (2S,4S,5S,7S)-5-Amino-4-hydroxy-2-isopropyl-7-[4-methoxy-3-(3-methoxy-propoxy)-benzyl]-8-methyl-nonanoic acid ((R)-tetrahydrofuran-3-yl)-amide

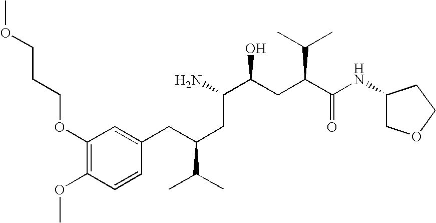

The title compound is prepared in accordance to Example 1. In contrast to cleavage of both the silyl and the Boc protecting groups simultaneously by the action of 4N HCl/dioxane (as described in Example 1) the silyl group is cleaved first (TBAF, THF, RT) followed by cleavage of the Boc group (ZnBr₂, 1,2-dichloroethane, 50° C.) as described in Example 9. t_{R} (HPLC, C18 column, 10-100% CH₃CN/H₂O/5 min, 100% CH₃CN/3 min, 100-10% CH₃CN/H₂O/3 min, flow: 1.5 ml/min): 4.51 min. MS (LC-MS): 523.1 [M+H]⁺.

### Example 13

### (2S,4S,5S,7S)-5-Amino-4-hydroxy-2-isopropyl-7-[4-methoxy-3-(3-methoxy-propoxy)-benzyl]-8-methyl-nonanoic acid ((S)-tetrahydrofuran-3-yl)-amide

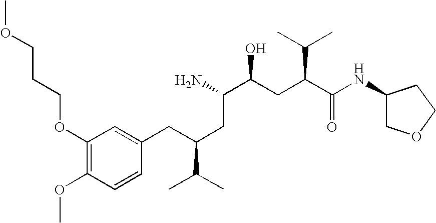

The title compound is prepared in accordance to Example 1. In contrast to cleavage of both the silyl and the Boc protecting groups simultaneously by the action of 4N HCl/dioxane (as described in Example 1) the silyl group is cleaved first (TBAF, THF, RT) followed by cleavage of the Boc group (ZnBr₂, 1,2-dichloroethane, 50° C.) as described in Example 9. t_{R} (HPLC, C18 column, 10-100% CH₃CN/H₂O/5 min, 100% CH₃CN/3 min, 100-10% CH₃CN/H₂O/3 min, flow: 1.5 ml/min): 4.58 min. MS (LC-MS): 523.1 [M+H]⁺.

The starting material is prepared as follows:

a) (S)-(Tetrahydro-furan-3-yl)amine is prepared according to Liebigs Ann. Chem. 1998, 1127.

### Example 14

### (2S,4S,5S,7S)-5-Amino-4-hydroxy-2-isopropyl-7-[4-methoxy-3-(3-methoxy-propoxy)-benzyl]-8-methyl-nonanoic acid (tetrahydro-pyran-4-ylmethyl)-amide

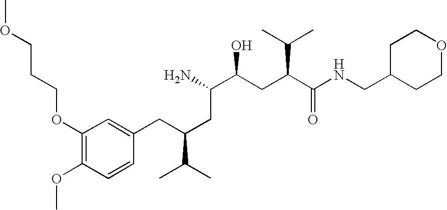

The title compound is prepared in accordance to Example 1. t_{R} (HPLC, C18 column, 10-100% CH₃CN/H₂O/5 min, 100% CH₃CN/3 min, 100-10% CH₃CN/H₂O/3 min, flow: 1.5 ml/min): 4.70 min. MS (LC-MS): 551.3 [M+H]⁺.

### Example 15

### (2S,4S,5S,7S)-5-Amino-4-hydroxy-2-isopropyl-7-[4-methoxy-3-(3-methoxy-propoxy)-benzyl]-8-methyl-nonanoic acid ([1,3]dioxolan-2-ylmethyl)-amide

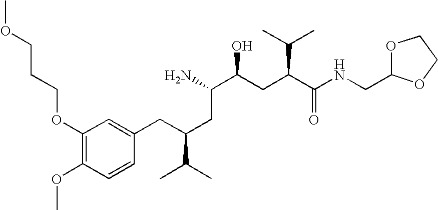

The title compound is prepared in accordance to Example 2. t_{R} (HPLC, C8 column, 5-95% CH₃CN/H₂O/6.5 min, 95% CH₃CN/H₂O/1 min, flow: 0.5 ml/min): 4.22 min. MS (LC-MS): 540.0 [M+H]⁺.

### Example 16

### (2S,4S,5S,7S)-5-Amino-4-hydroxy-2-isopropyl-7-[4-methoxy-3-(3-methoxy-propoxy)-benzyl]-8-methyl-nonanoic acid (2,2-dimethyl-[1,3]dioxan-5-ylmethyl)-amide

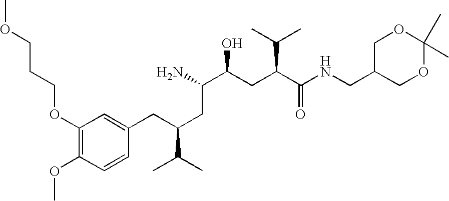

The title compound is prepared in accordance to Example 2. t_{R} (HPLC, C8 column, 5-95% CH₃CN/H₂O/6.5 min, 95% CH₃CN/H₂O/1 min, flow: 0.5 ml/min): 5.07 min. MS (LC-MS): 582 [MH]⁺.

### Example 17

### (2S,4S,5S,7S)-5-Amino-4-hydroxy-2-isopropyl-7-[4-methoxy-3-(3-methoxy-propoxy)-benzyl]-8-methyl-nonanoic acid (furan-3-ylmethyl)-amide

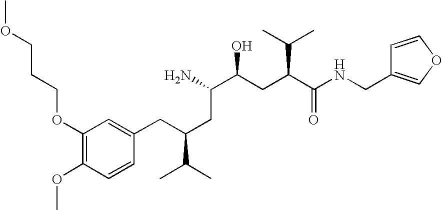

The title compound is prepared in accordance to Example 1. In contrast to cleavage of both the silyl and the Boc protecting groups simultaneously by the action of 4N HCl/dioxane (as described in Example 1), the silyl group is cleaved first (TBAF, THF, RT) followed by cleavage of the Boc group (4N HCl dioxane), as described in Example 4: t_{R} (HPLC, C18 column, 10-100% CH₃CN/H₂O/5 min, 100% CH₃CN/3 min, 100-10% CH₃CN/H₂O/3 min, flow: 1.5 ml/min): 4.88 min. MS (LC-MS): 533.1 [M+H]⁺.

### Example 18

### (2S,4S,5S,7S)-5-Amino-4-hydroxy-2-isopropyl-7-[4-methoxy-3-(3-methoxy-propoxy)-benzyl]-8-methyl-nonanoic acid (furan-2-ylmethyl)-amide

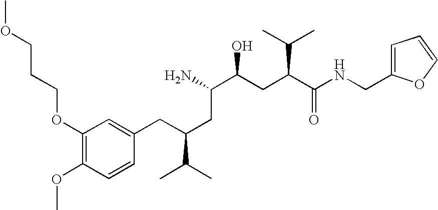

The title compound is prepared in accordance to Example 1. t_{R} (HPLC, C18 column, 10-100% CH₃CN/H₂O/5 min, 100% CH₃CN/3 min, 100-10% CH₃CN/H₂O/3 min, flow: 1.5 ml/min): 4.89 min. MS (LC-MS): 533.3 [M+H]⁺.

### Example 19

### (2S,4S,5S,7S)-5-Amino-4-hydroxy-2-isopropyl-7-[4-methoxy-3-(3-methoxy-propoxy)-benzyl]-8-methyl-nonanoic acid [2(R) or 2(S)-1-(tetrahydro-pyran-2-yl)methyl]-amide

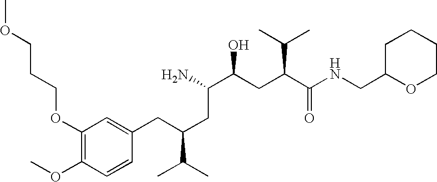

The solution of (2S,4S,5S,7S)-5-azido-4-hydroxy-2-isopropyl-7-[4-methoxy-3-(3-methoxy-propoxy)-benzyl]-8-methyl-nonanoic acid [2(R) or 2(S)-2-(tetrahydro-pyran-2-ylmethyl)]-amide (501 mg, 0.87 mmol) in a 4:1-mixture of tert-butylmethylether and MeOH (20 mL) is hydrogenated in the presence of Pd/C 10% (Engelhard 40708; 175 mg) and ethanolamine (0.16 mL) for 20 h at 25° C. and under 1 atm. The mixture is filtered through Celite®, followed by washing with EtOH and evaporation of the combined filtrates to afford the title compound as its free base, which is dissolved in isopropanol (3.0 mL) and a 0.1 M solution of fumaric acid in isopropanol (3.87 mL) is added. The solvent is removed in vacuo and the residue is freeze-dried from dioxane to give the title compound as its hemi-fumarate salt (off-white powder). RP-HPLC: t_{R}=4.68 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 551.4.

The starting material is prepared as follows:

### a) (2S,4S,5S,7S)-5-Azido-4-hydroxy-2-isopropyl-7-[4-methoxy-3-(3-methoxy-propoxy)-benzyl]-8-methyl-nonanoic acid [2(R) or 2(S)-2-(tetrahydro-pyran-2-ylmethyl)]-amide

The title compound is obtained by chromatographic separation of the diastereomeric mixture of (2S,4S,5S,7S)-5-azido-4-hydroxy-2-isopropyl-7-[4-methoxy-3-(3-methoxy-propoxy)-benzyl]-8-methyl-nonanoic acid [(R)- and (S)-2-(tetrahydro-pyran-2-ylmethyl)]-amide (1.38 g) on a Chiralpak AD® preparative column. Yellowish oil. Diastereomeric purity de>99.9% by analytical chiral HPLC on Chiralpak AD-H® (particle size: 5 μm; column dimensions: 0.46×25 cm; eluent: n-hexane/EtOH 95:5; flow rate: 1 mL/min) with t_{R}=24.5 min (first eluting diastereomer). RP-HPLC: t_{R}=6.04 min (Nucleosil C18HD column, 5-100% CH₃CN/H₂O/6 min, 100% CH₃CN/1.5 min, CH₃CN and H₂O containing 0.1% TFA, flow: 1.0 mL/min).

### b) (2S,4S,5S,7S)-5-Azido-4-hydroxy-2-isopropyl-7-[4-methoxy-3-(3-methoxy-propoxy)-benzyl]-8-methyl-nonanoic acid (tetrahydro-pyran-2-ylmethyl)-amide

The mixture of (3S,5S)-5-{(1S,3S)-1-azido-3-[4-methoxy-3-(3-methoxy-propoxy)-benzyl]-4-methyl-pentyl}-3-isopropyl-dihydro-furan-2-one [324763-46-4] (1.0 g, 2.17 mmol), racemic (tetrahydro-pyran-2-yl)-methylamine [6628-83-7] (2.50 g, 21.7 mmol) and AcOH (2 μL) is heated at 55° C. for 18 h. After cooling, the mixture is concentrated under reduced pressure and the residue is purified by FC on silica gel (CH₂Cl₂/acetone 97:3, then CH₂Cl₂/MeOH 97:3) to give the title compound as an oil. RP-HPLC: t_{R}=6.05 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 577.4.

### Example 20

### (2S,4S,5S,7S)-5-Amino-4-hydroxy-2-isopropyl-7-[4-methoxy-3-(3-methoxy-propoxy)-benzyl]-8-methyl-nonanoic acid [(R)- or (S)-1-(tetrahydro-pyran-2-yl)methyl]-amide

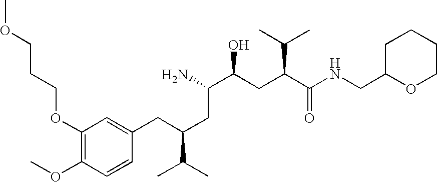

The solution of (2S,4S,5S,7S)-5-azido-4-hydroxy-2-isopropyl-7-[4-methoxy-3-(3-methoxy-propoxy)-benzyl]-8-methyl-nonanoic acid [(R)- or (S)-2-(tetrahydro-pyran-2-ylmethyl)]-amide (402 mg, 0.70 mmol) in a 4:1-mixture of tert-butylmethylether and MeOH (20 mL) is hydrogenated in the presence of Pd/C 10% (Engelhard 40708; 140 mg) and ethanolamine (0.16 mL) for 20 h at 25° C. under 1 atm. The mixture is filtered through Celite®, followed by washing with EtOH and evaporation of the combined filtrates to afford the title compound as its free base, which is dissolved in isopropanol (3.0 mL) and a 0.1 M solution of fumaric acid in isopropanol (3.27 mL) is added. The solvent is removed in vacuo and the residue is freeze-dried from dioxane to give the title compound as its hemi-fumarate salt (off-white powder). RP-HPLC: t_{R}=4.74 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 551.4.

The starting material is prepared as follows:

### a) (2S,4S,5S,7S)-5-Azido-4-hydroxy-2-isopropyl-7-[4-methoxy-3-(3-methoxy-propoxy)-benzyl]-8-methyl-nonanoic acid [(R)- or (S)-2-(tetrahydro-pyran-2-ylmethyl)]-amide

The title compound is obtained by chromatographic separation of the diastereomeric mixture of (2S,4S,5S,7S)-5-azido-4-hydroxy-2-isopropyl-7-[4-methoxy-3-(3-methoxy-propoxy)-benzyl]-8-methyl-nonanoic acid [(R)- and (S)-2-(tetrahydro-pyran-2-ylmethyl)]-amide (1.38 g) on a Chiralpak AD® preparative column. Yellowish oil. Diastereomeric purity de>99.0% by analytical chiral HPLC on Chiralpak AD-H® (particle size: 5 μm; column dimensions: 0.46×25 cm; eluent: n-hexane/EtOH 95:5; flow rate: 1 mL/min) with t_{R}=29.0 min (second eluting diastereomer). RP-HPLC: t_{R}=6.04 min (Nucleosil C18HD column, 5-100% CH₃CN/H₂O/6 min, 100% CH₃CN/1.5 min, CH₃CN and H₂O containing 0.1% TFA, flow: 1.0 mL/min).

### Example 21

### (2S,4S,5S,7S)-5-Amino-4-hydroxy-2-isopropyl-7-[4-methoxy-3-(3-methoxy-propoxy)-benzyl]-8-methyl-nonanoic acid (4-hydroxy-tetrahydro-pyran-4-ylmethyl)-amide

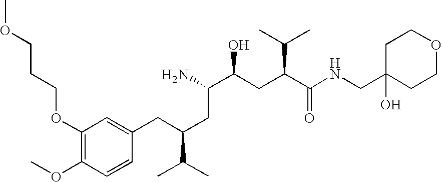

The solution of (2S,4S,5S,7S)-5-azido-4-hydroxy-2-isopropyl-7-[4-methoxy-3-(3-methoxy-propoxy)-benzyl]-8-methyl-nonanoic acid (4-hydroxy-tetrahydro-pyran-4-ylmethyl)-amide (760 mg, 1.28 mmol) in a 4:1-mixture of tert-butylmethylether and MeOH (40 mL) is hydrogenated in the presence of Pd/C 10% (Engelhard 40708; 250 mg) and ethanolamine (0.25 mL) for 17 h at 25° C. under 1 atm. The mixture is filtered through Celite®, followed by washing with EtOH and evaporation of the combined filtrates. The product is purified by FC on silica gel (CH₂Cl₂/MeOH/NH₃ conc. (10%) 97:3, then CH₂Cl₂/MeOH/NH₃ conc. (10%) 90:10) to afford the title compound as its free base, which is dissolved in isopropanol (3.0 mL) and a 0.1 M solution of fumaric acid in isopropanol (5.38 mL) is added. The solvent is removed in vacuo and the residue is freeze-dried from dioxane to give the title compound as its hemi-fumarate salt (off-white powder). RP-HPLC: t_{R}=4.55 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 567.4.

The starting material is prepared as follows:

### a) (2S,4S,5S,7S)-5-azido-4-hydroxy-2-isopropyl-7-[4-methoxy-3-(3-methoxy-proloxy)-benzyl]-8-methyl-nonanoic acid (4-hydroxy-tetrahydro-pyran-4-ylmethyl)-amide

The title compound is obtained from (2S,4S,5S,7S)-5-azido-4-(tert-butyl-dimethyl-silanyloxy)-2-isopropyl-7-[4-methoxy-3-(3-methoxy-propoxy)-benzyl]-8-methyl-nonanoic acid (4-hydroxy-tetrahydro-pyran-4-ylmethyl)-amide (1.04 g, 1.47 mmol) and reaction with tetrabutyl ammoniumfluoride trihydrate (696 mg, 2.21 mmol) in THF (10 mL) as described in Example 2. The product is purified by FC on silica gel (n-hexane/EtOAc 25:75, then EtOAc 100%) to give a colorless oil. RP-HPLC: t_{R}=5.38 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 593.4.

b) (2S,4S,5S,7S)-5-azido-4-(tert-butyl-dimethyl-silanyloxy)-2-isopropyl-7-[4-methoxy-3-(3-methoxy-propoxy)-benzyl]-8-methyl-nonanoic acid (4-hydroxy-tetrahydro-pyran-4-ylmethyl)-amide

To a solution of (2S,4S,5S,7S)-5-azido-4-(tert-butyl-dimethyl-silanyloxy)-2-isopropyl-7-[4-methoxy-3-(3-methoxy-propoxy)-benzyl]-8-methyl-nonanoic acid (1.50 g, 2.53 mmol) in acetonitrile (15 mL) is added HBTU (1.15 g, 3.03 mmol) and, after stirring for 5 min, a solution of 4-aminomethyl-tetrahydro-pyran-4-ol hydrochloride salt (847 mg, 5.05 mmol) and NEt₃ (3.52 mL, 25.3 mmol) in acetonitrile (15 mL) is added to the mixture. After stirring the mixture overnight at RT, volatiles are removed under reduced pressure. The residue is taken up in CH₂Cl₂ and the organics are successively washed with saturated aqueous NH₄Cl solution and saturated aqueous NaHCO₃, dried over MgSO₄ and concentrated in vacuo. The product is purified by FC on silica gel (n-hexane/EtOAc 7:3, then n-hexane/EtOAc 25:75) to give the title compound as colorless oil. RP-HPLC: t_{R}=7.23 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M]⁺707.4.

### c) 4-Aminomethyl-tetrahydro-pyran-4-ol hydrochloride salt

To a 1M solution of LiAlH₄ in THF (98.3 mL) is added 4-hydroxy-tetrahydro-pyran-4-carbonitrile [50289-10-6] (2.50 g, 19.7 mmol; prepared according to Archiv der Pharmazie (Weinheim, Germany) 1987, 320, 348-61) in a dropwise manner under argon. The mixture is refluxed with stirring for 4 h, then cooled to RT and then quenched by dropwise addition of water (5 mL) and 2M NaOH (5 mL). The white suspension is filtered through Celite®, followed by washing with diethylether. To the combined filtrates is added 4M HCl in dioxane (1.5 equivalents) to give, after evaporation of solvents and drying in vacuo, the title compound as off-white solid. MS: [M+H]⁺ 132.2.

### Example 22

### (2S,4S,5S,7S)-5-Amino-4-hydroxy-2-isopropyl-7-[4-methoxy-3-(3-methoxy-propoxy)-benzyl]-8-methyl-nonanoic acid [(R)- or (S)-3-(tetrahydro-pyran-3-yl)]-amide

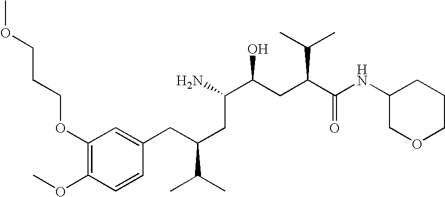

The solution of (2S,4S,5S,7S)-5-azido-4-hydroxy-2-isopropyl-7-[4-methoxy-3-(3-methoxy-propoxy)-benzyl]-8-methyl-nonanoic acid [(R)- or (S)-3-(tetrahydro-pyran-3-yl)-amide (735 mg, 1.31 mmol) in a 4:1-mixture of tert-butylmethylether and MeOH (30 mL) is hydrogenated in the presence of Pd/C 10% (Engelhard 40708; 260 mg) and ethanolamine (0.25 mL) for 20 h at 25° C. under 1 atm. The mixture is filtered through Celite®, followed by washing with EtOH and evaporation of the combined filtrates to afford the title compound as its free base, which is dissolved in isopropanol (5.0 mL) and a 0.1 M solution of fumaric acid in isopropanol (4.53 mL) is added. The solvent is removed in vacuo and the residue is freeze-dried from dioxane to give the title compound as its hemi-fumarate salt (off-white powder). RP-HPLC: t_{R}=4.51 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 537.4.

The starting material is prepared as follows:

### a) (2S,4S,5S,7S)-5-Azido-4-hydroxy-2-isopropyl-7-[4-methoxy-3-(3-methoxy-propoxy)-benzyl]-8-methyl-nonanoic acid F(R)- or (S)-3-(tetrahydro-pyran-3-yl)-amide

The title compound is obtained by chromatographic separation of the diastereomeric mixture of (2S,4S,5S,7S)-5-azido-4-hydroxy-2-isopropyl-7-[4-methoxy-3-(3-methoxy-propoxy)-benzyl]-8-methyl-nonanoic acid [(R) and (S)-3-(tetrahydro-pyran-3-yl)-amide (1.8 g) on a Chiralpak AD® column (dimension: 3.0×25 cm; mobile phase A: CO₂/180 bar/30° C.; mobile phase B: EtOH; isocratic eluent: 10% B; flow rate: 120 g/min). Yellowish oil. Diastereomeric purity de>99.9% by analytical chiral HPLC on Chiralpak AD® (column dimensions: 0.46×25 cm; eluent: n-hexane/EtOH 95:5; flow rate: 1 mL/min) with t_{R}=14.3 min (first eluting diastereomer). RP-HPLC: t_{R}=5.76 min (Nucleosil C18HD column, 5-100% CH₃CN/H₂O/6 min, 100% CH₃CN/1.5 min, CH₃CN and H₂O containing 0.1% TFA, flow: 1.0 mL/min).

### b) (2S,4S,5S,7S)-5-Azido-4-hydroxy-2-isopropyl-7-[4-methoxy-3-(3-methoxy-propoxy)-benzyl]-8-methyl-nonanoic acid f(R) and (S)-3-(tetrahydro-pyran-3-yl)-amide

The mixture of (3S,5S)-5-{(1S,3S)-1-azido-3-[4-methoxy-3-(3-methoxy-propoxy)-benzyl]-4-methyl-pentyl}-3-isopropyl-dihydro-furan-2-one (2.0 g, 4.33 mmol), racemic tetrahydro-pyran-3-ylamine [120811-32-7] (3.07 g, 30.3 mmol) and AcOH (2 μL) is heated at 70° C. for 3 days. After cooling to RT, the mixture is concentrated under reduced pressure and the residue is purified by FC on silica gel (CH₂Cl₂/acetone 97:3, then CH₂Cl₂/MeOH 97:3) to give the title compound as yellowish oil. MS: [M+H]⁺ 563.4.

### Example 23

### (2S,4S,5S,7S)-5-Amino-4-hydroxy-2-isopropyl-7-[4-methoxy-3-(3-methoxy-propoxy)-benzyl]-8-methyl-nonanoic acid [(R)- or (S)-3-(tetrahydro-pyran-3-yl)]-amide

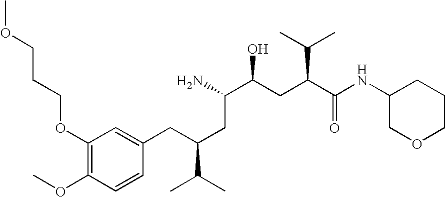

The solution of (2S,4S,5S,7S)-5-azido-4-hydroxy-2-isopropyl-7-[4-methoxy-3-(3-methoxy-propoxy)-benzyl]-8-methyl-nonanoic acid [(R)- or (S)-3-(tetrahydro-pyran-3-yl)-amide (688 mg, 1.22 mmol) in a 4:1-mixture of tert-butylmethylether and MeOH (30 mL) is hydrogenated in the presence of Pd/C 10% (Engelhard 40708; 250 mg) and ethanolamine (0.235 mL) overnight at 25° C. under 1 atm. The mixture is filtered through Celite®, followed by washing with EtOH and evaporation of the combined filtrates to afford the title compound as its free base, which is dissolved in isopropanol (5.0 mL) and a 0.1 M solution of fumaric acid in isopropanol (3.47 mL) is added. The solvent is removed in vacuo and the residue is freeze-dried from dioxane to give the title compound as its hemi-fumarate salt (off-white powder). RP-HPLC: t_{R}=4.54 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 537.4.

The starting material is prepared as follows:

### a) (2S,4S,5S,7S)-5-Azido-4-hydroxy-2-isopropyl-7-[4-methoxy-3-(3-methoxy-propoxy)-benzyl]-8-methyl-nonanoic acid [(R)- or (S)-3-(tetrahydro-pyran-3-yl)-amide

The title compound is obtained by chromatographic separation of the diastereomeric mixture of (2S,4S,5S,7S)-5-azido-4-hydroxy-2-isopropyl-7-[4-methoxy-3-(3-methoxy-propoxy)-benzyl]-8-methyl-nonanoic acid [(R) and (S)-3-(tetrahydro-pyran-3-yl)-amide (1.8 g) on a Chiralpak AD® column (dimension: 3.0×25 cm; mobile phase A: CO₂/180 bar/30° C.; mobile phase B: EtOH; isocratic eluent: 10% B; flow rate: 120 g/min). Yellowish oil. Diastereomeric purity de>98.3% by analytical chiral HPLC on Chiralpak AD® (column dimensions: 0.46×25 cm; eluent: n-hexane/EtOH 95:5; flow rate: 1 mL/min) with t_{R}=20.1 min (second eluting diastereomer). RP-HPLC: t_{R}=5.73 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 563.4.

### Example 24

### (2S,4S,5S,7S)-5-Amino-4-hydroxy-2-isopropyl-7-[4-methoxy-3-(3-methoxy-propoxy)-benzyl]-8-methyl-nonanoic acid [trans-(3S,4R)- or [(3R,4S)-4-hydroxy-tetrahydro-furan-3-yl]-amide

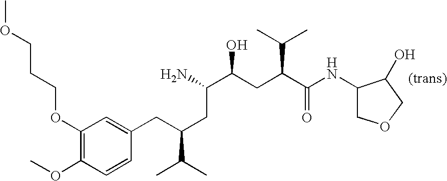

The solution of (2S,4S,5S,7S)-5-Amino-4-hydroxy-2-isopropyl-7-[4-methoxy-3-(3-methoxy-propoxy)-benzyl]-8-methyl-nonanoic acid [trans-(3S,4R)- or (3R,4S)-4-hydroxy-tetrahydro-furan-3-yl]-amide (710 mg, 1.26 mmol) in a 4:1-mixture of tert-butylmethylether and MeOH (50 mL) is hydrogenated in the presence of Pd/C 10% (Engelhard 40708; 240 mg) and ethanolamine (0.24 mL) for 16 h at 25° C. under 1 atm. The mixture is filtered through Celite®, followed by washing with EtOH and evaporation of the combined filtrates. The product is purified by FC on silica gel (CH₂Cl₂/MeOH/NH₃ conc. (10%) 97:3, then CH₂Cl₂/MeOH/NH₃ conc. (10%) 85:15) to give the title compound as its free base, which is dissolved in isopropanol (2.0 mL) and a 0.1 M solution of fumaric acid in isopropanol (4.83 mL) is added. The solvent is removed in vacuo and the residue is freeze-dried from dioxane to give the title compound as its hemi-fumarate salt (off-white powder). RP-HPLC: t_{R}=4.16 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 539.4.

The starting material is prepared as follows:

### a) (2S,4S,5S,7S)-5-Azido-4-hydroxy-2-isopropyl-7-[4-methoxy-3-(3-methoxy-propoxy)-benzyl]-8-methyl-nonanoic acid [trans-(3S,4R)- or (3R,4S)-4-hydroxy-tetrahydro-furan-3-yl]-amide

The title compound is obtained by chromatographic separation of the trans-diastereomeric mixture of (2S,4S,5S,7S)-5-azido-4-hydroxy-2-isopropyl-7-[4-methoxy-73-(3-methoxy-propoxy)-benzyl]-8-methyl-nonanoic acid [(3S,4R)- and (3R,4S)-4-hydroxy-tetrahydro-furan-3-yl]-amide (1.7 g) on a Chiralpak AD® preparative column. Yellowish oil. Diastereomeric purity de>99.9% by analytical chiral HPLC on Chiralpak AD-H® (particle size: 5 μm; column dimensions: 0.46×25 cm; eluent: n-hexane/EtOH 9:1; flow rate: 1 mL/min) with t_{R}=8.83 min (first eluting diastereomer). RP-HPLC: t_{R}=5.25 min (Nucleosil C18HD column, 5-100% CH₃CN/H₂O/6 min, 100% CH₃CN/1.5 min, CH₃CN and H₂O containing 0.1% TFA, flow: 1.0 mL/min).

### b) (2S,4S,5S,7S)-5-Azido-4-hydroxy-2-isopropyl-7-[4-methoxy-3-(3-methoxy-propoxy)-benzyl]-8-methyl-nonanoic acid [(3S,4R)- and (3R,4S)-4-hydroxy-tetrahydro-furan-3-yl]-amide

The mixture of (3S,5S)-5-{(1S,3S)-1-azido-3-[4-methoxy-3-(3-methoxy-propoxy)-benzyl]-4-methyl-pentyl}-3-isopropyl-dihydro-furan-2-one (2.0 g, 4.33 mmol), racemic trans-4-amino-tetrahydro-furan-3-ol (2.23 g, 21.7 mmol; prepared according to Journal of Medicinal Chemistry (2001), 44, 725-736) and AcOH (2 μL) is heated at 80° C. for 3 days. The product is purified by FC on silica gel (CH₂Cl₂/MeOH gradient 100:0 to 85:15) to give the title compound as an oil. RP-HPLC: t_{R}=5.24 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 565.3.

### Example 25

### (2S,4S,5S,7S)-5-Amino-4-hydroxy-2-isopropyl-7-[4-methoxy-3-(3-methoxy-propoxy)-benzyl]-8-methyl-nonanoic acid [trans-(3R,4S)- or (3S,4R)-4-hydroxy-tetrahydro-furan-3-yl]-amide

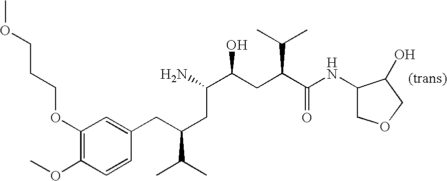

The title compound is obtained from (2S,4S,5S,7S)-5-azido-4-hydroxy-2-isopropyl-7-[4-methoxy-3-(3-methoxy-propoxy)-benzyl]-8-methyl-nonanoic acid [trans-(3S,4R)- or (3R,4S)-4-hydroxy-tetrahydro-furan-3-yl]-amide (840 mg, 1.49 mmol) according to the procedure described in Example 24 as its hemi-fumarate salt (off-white powder). RP-HPLC: t_{R}=4.11 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 539.4.

The starting material is prepared as follows:

### a) (2S,4S,5S,7S)-5-Azido-4-hydroxy-2-isopropyl-7-[4-methoxy-3-(3-methoxy-propoxy)-benzyl]-8-methyl-nonanoic acid [trans-(3S,4R)- or (3R,4S)-4-hydroxy-tetrahydro-furan-3-yl]-amide

The title compound is obtained by chromatographic separation of the trans-diastereomeric mixture of (2S,4S,5S,7S)-5-azido-4-hydroxy-2-isopropyl-7-[4-methoxy-3-(3-methoxy-propoxy)-benzyl]-8-methyl-nonanoic acid [(3S,4R)- and (3R,4S)-4-hydroxy-tetrahydro-furan-3-yl]-amide (1.7 g) on a Chiralpak ADO preparative column. Yellowish oil. Diastereomeric purity de>98.2% by analytical chiral HPLC on Chiralpak AD-H® (particle size: 5 μm; column dimensions: 0.46×25 cm; eluent: n-hexane/EtOH 9:1; flow rate: 1 mL/min) with t_{R}=11.0 min (second eluting diastereomer). RP-HPLC: t_{R}=5.24 min (Nucleosil C18HD column, 5-100% CH₃CN/H₂O/6 min, 100% CH₃CN/1.5 min, CH₃CN and H₂O containing 0.1% TFA, flow: 1.0 mL/min).

### Example 26

### (2S,4S,5S,7S)-5-Amino-4-hydroxy-2-isopropyl-7-[3-(3-methoxy-propoxy)-4-methyl-benzyl]-8-methyl-nonanoic acid (tetrahydro-pyran-4-yl)-amide

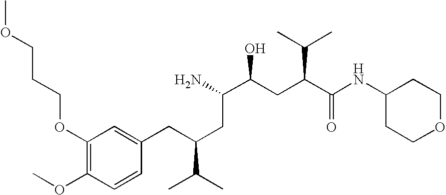

To a solution of [(1S,2S,4S)-2-hydroxy-1-{(S)-2-[3-(3-methoxy-propoxy)-4-methyl-benzyl]-3-methyl-butyl}-5-methyl-4-(tetrahydro-pyran-4-ylcarbamoyl)-hexyl]-carbamic acid tert-butyl ester (455 mg, 0.73 mmol) in dioxane (2.0 mL), cooled to 0° C., is added 4M HCl in dioxane (5.8 mL) and stirring is continued at 5 to 10° C. for 6 h. Volatiles are removed by freeze-drying in high vacuo. The product is purified by FC on silca gel (CH₂Cl₂/MeOH/conc. NH₃ (10%) 93:7) to afford the title compound as free base, which is dissolved in isopropanol (1.5 mL) and a 0.1 M solution of fumaric acid in isopropanol (3.12 mL) is added. The solvent is removed in vacuo and the residue is freeze-dried to give the title compound as its hemi-fumarate salt (off-white powder). RP-HPLC: t_{R}=5.04 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 521.4.

The starting material is prepared as follows:

### a) (1S,2S,4S)-2-Hydroxy-1-{(S)-2-[3-(3-methoxy-propoxy)-4-methyl-benzyl]-3-methyl-butyl}-5-methyl-4-(tetrahydro-pyran-4-ylcarbamoyl)-hexyl]-carbamic acid tert-butyl ester

The title compound is prepared from {(1S,3S)-1-((S)-4-isopropyl-5-oxo-tetrahydro-furan-2-yl)-3-[3-(3-methoxy-propoxy)-4-methyl-benzyl]-4-methyl-pentyl}-carbamic acid tert-butyl ester (540 mg, 1.04 mmol) and tetrahydro-pyran-4-ylamine (1.05 g, 10.4 mmol) in the presence of AcOH (2 μL) as described in Example 22a). The product is purified by FC on silica gel (CH₂Cl₂/acetone 97:3, then CH₂Cl₂/MeOH 97:3) to give a yellowish oil. RP-HPLC: t_{R}=6.24 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 621.4.

### b) {(1S,3S)-1-((2S,4S)-4-isopropyl-5-oxo-tetrahydro-furan-2-yl)-3-[3-(3-methoxy-propoxy)-4-methyl-benzyl]-4-methyl-pentyl}-carbamic acid tert-butyl ester

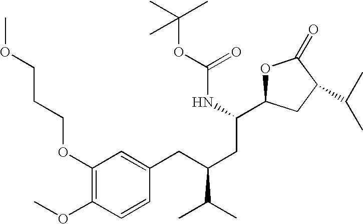

A solution of (3S,5S)-5-{(1S,3S)-1-amino-3-[3-(3-methoxy-propoxy)-4-methyl-benzyl]-4-methyl-pentyl}-3-isopropyl-dihydro-furan-2-one (2.40 g, 5.72 mmol), di-tert-butyl dicarbonate (1.37 g, 6.29 mmol) and NEt₃ (0.88 mL, 6.29 mmol) in CH₂Cl₂ (40 mL) is stirred for 18 hours at RT. The mixture is diluted by adding 100 mL of CH₂Cl₂, and the organic layer is washed with 1M HCl (50 mL), saturated NaHCO₃ and brine, dried (Na₂SO₄) and concentrated in vacuo. The residue is purified by FC on silica gel (CH₂Cl₂/acetone gradient 100:0 to 97:3) to afford the title compound as colorless oil. RP-HPLC: t_{R}=6.82 min (Nucleosil C-18HD column, 20-100% CH₃CN/H₂O/6 min, 100% CH₃CN/1.5 min, CH₃CN and H₂O containing 0.1% TFA, flow: 1.0 mL/min). MS: [M+H₂O]⁺537.4.

### c) (3S,5S)-5-{(1S,3S)-1-Amino-3-[3-(3-methoxy-propoxy)-4-methyl-benzyl]-4-methyl-pentyl}-3-isopropyl-dihydro-furan-2-one

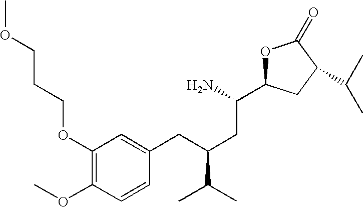

A solution of isobutyric acid (S)-2-[(S)-2-azido-2-((2S,4S)-4-isopropyl-5-oxo-tetrahydro-furan-2-yl)-ethyl]-1-[3-(3-methoxy-propoxy)-4-methyl-phenyl]-3-methyl-butyl ester (3.00 g, 5.64 mmol) and ethanolamine (0.39 g, 6.21 mmol) in EtOH (150 mL) is hydrogenated over palladium on carbon (10%, 6.0 g; Engelhardt 40708) at 25° C. under 1 atm overnight. The mixture is filtered through Celite®, washed with EtOH and the combined filtrates are concentrated to afford the crude title compound as a colorless oil. R_{f} (CH₂Cl₂MeOH/conc.NH₃(10%) 9:1): 0.80. RP-HPLC: t_{R}=5.34 min (Nucleosil C-18HD column, 20-100% CH₃CN/H₂O/6 min, 100% CH₃CN/1.5 min, CH₃CN and H₂O containing 0.1% TFA, flow: 1.0 mL/min). MS: [M+H₂O]⁺420.2.

### d) Isobutyric acid (S)-2-[(S)-2-azido-2-((2S,4S)-4-isopropyl-5-oxo-tetrahydro-furan-2-yl)-ethyl]-1-[3-(3-methoxy-propoxy)-4-methyl-phenyl]-3-methyl-butyl ester

A solution of (3S,5S)-5-((1S,3S)-1-azido-3-{hydroxy-[3-(3-methoxy-propoxy)-4-methyl-phenyl]-methyl}-4-methyl-pentyl)-3-isopropyl-dihydro-furan-2-one (6.89 g, 13.6 mmol), pyridine (5.47 mL, 67.9 mmol), 4-(N,N-dimethylamino)-pyridine (332 mg, 2.72 mmol) and isobutyric anhydride (6.76 mL, 40.7 mmol) in CH₂Cl₂ (150 mL) is stirred at RT for 1.5 hours. Volatiles are evaporated in vacuo, the residue is taken up in EtOAc (250 mL) and subsequently washed with water, 0.5M HCl water, saturated aqueous NaHCO₃ and brine (each 100 mL). The organic layer is dried over Na₂SO₄, concentrated and the crude product is purified by FC on silica gel (gradient CH₂Cl₂ to CH₂Cl₂/acetone 98:2) to give the title compound as a colorless oil. R_{f} (CH₂Cl₂/acetone 98:2): 0.47. RP-HPLC: t_{R}=6.75 and 6.82 min (Nucleosil C-18HD column, 20-100% CH₃CN/H₂O/6 min, 100% CH₃CN/1.5 min, CH₃CN and H₂O containing 0.1% TFA, flow: 1.0 mL/min). MS: [M+H₂O]⁺549.4.

### e) (3S,5S)-5-((1 S,3S)-1-Azido-3-{hydroxy-[3-(3-methoxy-propoxy)-4-methyl-phenyl]-methyl}-4-methyl-pentyl)-3-isopropyl-dihydro-furan-2-one

To a solution of 4-bromo-2-(3-methoxy-propoxy)-1-methyl-benzene (5.99 g, 23.1 mmol) and N-methylmorpholine (5.87 mL, 53.3 mmol) in dry THF (68 mL), cooled to −70 to −75° C. under an argon atmosphere, is added dropwise over 20 min a 1.6 M solution of n-butyl lithium in hexane (20.0 mL, 32.0 mmol). After stirring for 30 min, a solution of MgBr₂ in dry THF (170 mL), freshly prepared from Mg (1.30 g, 53.5 mmol) and 1,2-dibromoethane (4.61 mL, 53.5 mmol), is added dropwise over 15 min at −75° C. The mixture is stirred for 45 min, followed by dropwise addition of (S)-2-[(S)-2-azido-2-((2S,4S)-4-isopropyl-5-oxo-tetrahydro-furan-2-yl)-ethyl]-3-methyl-butyraldehyde (5.00 g, 17.8 mmol; prepared as described in EP0678503B1 and EP0678514A1) in THF (55 mL) over 20 min. After stirring for additional 30 min at −75° C., the reaction is quenched by adding a saturated aqueous solution of NH₄Cl (70 mL), followed by extraction of the water phase with EtOAc (3×200 mL). The combined organics are washed with a 1:1-mixture of brine and water (2×100 mL), dried over MgSO₄ and concentrated in vacuo. The product is purified by flash chromatography on silica gel (CH₂Cl₂/acetone gradient 98:2 to 95:5) to afford the title compound as a light yellow oil. R_{f} (CH₂Cl₂/acetone 98:2): 0.27. RP-HPLC: t_{R}=5.48 and 5.78 min (Nucleosil C-18HD column, 20-100% CH₃CN/H₂O/6 min, 100% CH₃CN/1.5 min, CH₃CN and H₂O containing 0.1% TFA, flow: 1.0 mL/min). MS: [M+H₂O]⁺479.2.

### f) 4-Bromo-2-(3-methoxy-propoxy)-1-methyl-benzene

The mixture of 5-bromo-2-methyl-phenol [36138-76-8] (11.5 g, 61.5 mmol), 1-bromo-3-methoxy-propane (14.1 g, 92.2 mmol; Matrix Scientific 007519) and anhydrous K₂CO₃ (12.7 g, 92.2 mmol) in acetonitrile (200 mL) is refluxed overnight with stirring. After cooling to RT, the mixture is filtered and the combined filtrates are concentrated in vacuo. Purification by flash chromatography on silica gel (n-hexane/EtOAc 95:5) gives the title compound as colorless oil. R_{f} (n-hexane/EtOAc 9:1): 0.68. R_{f} (CH₂Cl₂/acetone 98:2): 0.59. MS: [M]⁺259.0/261.0.

### Example 27

### (2S,4S,5S,7S)-5-Amino-4-hydroxy-2-isopropyl-7-[3-(3-methoxy-propoxy)-4-methyl-benzyl]-8-methyl-nonanoic acid [(R)-1-(tetrahydro-furan-2-yl)methyl]-amide

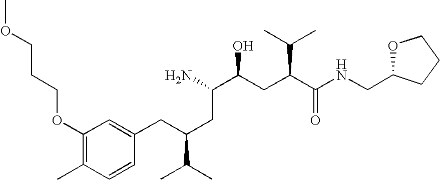

To a solution of ((1S,2S,4S)-2-hydroxy-1-{(S)-2-[3-(3-methoxy-propoxy)-4-methyl-benzyl]-3-methyl-butyl}-5-methyl-4-{[(R)-1-(tetrahydro-furan-2-yl)methyl]-carbamoyl}-hexyl)-carbamic acid tert-butyl ester (87 mg, 0.14 mmol) in dioxane (0.5 mL), cooled to 0° C., is added 4M HCl in dioxane (0.75 mL) and stirring is continued at 5 to 10° C. for 6 h. Volatiles are removed by freeze-drying in high vacuo. The product is purified by FC on silca gel (CH₂Cl₂/MeOH/conc. NH₃ (10%) 93:7) to afford the title compound as free base, which is dissolved in isopropanol (0.5 mL) and a 0.1 M solution of fumaric acid in isopropanol (0.52 mL) is added. The solvent is removed in vacuo, the residue is dissolved in dioxane followed by freeze-drying to afford the title compound as hemi-fumarate salt (off-white powder). RP-HPLC: t_{R}=5.39 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 521.4.

The starting material is prepared as follows:

### a) ((1S,2S,4S)-2-Hydroxy-1-{(S)-2-[3-(3-methoxy-propoxy)-4-methyl-benzyl]-3-methyl-butyl}-5-methyl-4-{[(R)-1-(tetrahydro-furan-2-yl)methyl]-carbamoyl}-hexyl)-carbamic acid tert-butyl ester

The mixture of {(1S,3S)-1-((S)-4-isopropyl-5-oxo-tetrahydro-furan-2-yl)-3-[3-(3-methoxy-propoxy)-4-methyl-benzyl]-4-methyl-pentyl}-carbamic acid tert-butyl ester (75 mg, 0.144 mmol) and [(R)-1-(tetrahydro-furan-2-yl)]-methylamine [7202-43-9] (150 μL, 1.44 mmol; Aldrich 41.293-1) is stirred at 50° C. in the presence of AcOH (0.5 μL) for 24 hours. After cooling, volatiles are removed under reduced pressure and the residue is purified by FC on silica gel (CH₂Cl₂/MeOH 97:3) to give the title compound as colorless oil. RP-HPLC: t_{R}=6.24 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 621.4.

### Example 28

### (2S,4S,5S,7S)-5-Amino-4-hydroxy-2-isopropyl-7-[3-(3-methoxy-propoxy)-4-methyl-benzyl]-8-methyl-nonanoic acid [(S)-1-(tetrahydro-furan-2-yl)methyl]-amide

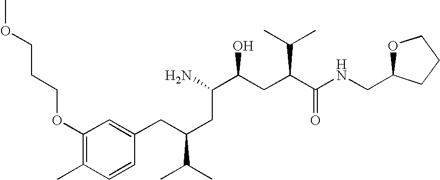

The title compound is prepared in a similar fashion as described in Example 27 from ((1S,2S,4S)-2-hydroxy-1-{(S)-2-[3-(3-methoxy-propoxy)-4-methyl-benzyl]-3-methyl-butyl}-5-methyl-4-{[(S)-1-(tetrahydro-furan-2-yl)methyl]-carbamoyl}-hexyl)-carbamic acid tert-butyl ester to afford the hemi-fumarate salt as off-white powder. RP-HPLC: t_{R}=5.41 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 521.4.

The starting material is prepared as follows:

### a) ((1S,2S,4S)-2-Hydroxy-1-{(S)-2-[3-(3-methoxy-propoxy)-4-methyl-benzyl]-3-methyl-butyl}-5-methyl-4-{[(S)-1-(tetrahydro-furan-2-yl)methyl]-carbamoyl}-hexyl)-carbamic acid tert-butyl ester

The title compound is prepared from {(1S,3S)-1-((S)-4-isopropyl-5-oxo-tetrahydro-furan-2-yl)-3-[3-(3-methoxy-propoxy)-4-methyl-benzyl]-4-methyl-pentyl}-carbamic acid tert-butyl ester (75 mg, 0.144 mmol) and [(S)-1-(tetrahydro-furan-2-yl)]-methylamine [7175-81-7] (150 μL, 1.44 mmol; Lancaster 10790) in the presence of AcOH (0.5 μL) as described in Example 27a). Colorless oil. RP-HPLC: t_{R}=6.23 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 621.4.

### Example 29

### (2S,4S,5S,7S)-5-Amino-4-hydroxy-2-isopropyl-7-[4-methoxy-3-(3-methoxy-propoxy)-benzyl]-8-methyl-nonanoic acid [3(R) or 3(S)-1-(tetrahydro-furan-3-yl)methyl]-amide

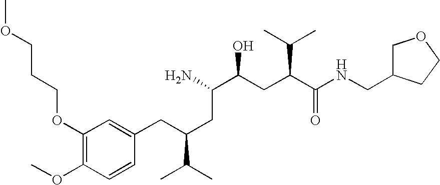

The solution of (2S,4S,5S,7S)-5-azido-4-hydroxy-2-isopropyl-7-[4-methoxy-3-(3-methoxy-propoxy)-benzyl]-8-methyl-nonanoic acid [3(R)- or 3(S)-(tetrahydro-furan-3-ylmethyl)-amide (453 mg, 0.805 mmol) in a 4:1-mixture of tert-butylmethylether and MeOH (20 mL) is hydrogenated in the presence of Pd/C 10% (Engelhard 40708; 160 mg) and ethanolamine (0.053 mL) overnight at 25° C. under 1 atm. The mixture is filtered through Celite®, followed by washing with EtOH and evaporation of the combined filtrates to give the product its free base, which is dissolved in isopropanol (3.0 mL) and a 0.1 M solution of fumaric acid in isopropanol (3.91 mL) is added. The solvent is removed in vacuo and the residue is freeze-dried from dioxane to give the title compound as its hemi-fumarate salt (off-white powder). RP-HPLC: t_{R}=4.36 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 537.4.

The starting material is prepared as follows:

### b) (2S,4S,5S,7S)-5-Azido-4-hydroxy-2-isopropyl-7-[4-methoxy-3-(3-methoxy-propoxy)-benzyl]-8-methyl-nonanoic acid [3(R)- or 3(S)-(tetrahydro-furan-3-ylmethyl)-amide

The title compound is obtained by chromatographic separation of the diastereomeric mixture of (2S,4S,5S,7S)-5-azido-4-hydroxy-2-isopropyl-7-[4-methoxy-3-(3-methoxy-propoxy)-benzyl]-8-methyl-nonanoic acid [3(R)- and 3(S)-(tetrahydro-furan-3-ylmethyl)-amide on a Chiralpak AD® preparative column (particle size: 20 μM; column dimensions: 5×20 cm; eluent: hexane/EtOH 9:1; flow rate 80 mL/min) on small scale. On larger scale separation is done on Chiralpak AD-i. Colorless oil. Diastereomeric purity de=99.9% by analytical chiral HPLC on Chiralpak AD-H® (column dimensions: 0.46×25 cm; eluent: n-hexane/EtOH 85:15; flow rate: 1 mL/min) with t_{R}=8.59 min (first eluting diastereomer). RP-HPLC: t_{R}=4.44 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 563.4.

### b) (2S,4S,5S,7S)-5-Azido-4-hydroxy-2-isopropyl-7-[4-methoxy-3-(3-methoxy-propoxy)-benzyl]-8-methyl-nonanoic acid [3(R)- and 3(S)-(tetrahydro-furan-3-ylmethyl)-amide

The mixture of (3S,5S)-5-{(1S,3S)-1-azido-3-[4-methoxy-3-(3-methoxy-propoxy)-benzyl]-4-methyl-pentyl}-3-isopropyl-dihydro-furan-2-one (0.50 g, 1.08 mmol), racemic C-(tetrahydro-furan-3-yl)-methylamine [165253-31-6] (1.10 g, 10.8 mmol; Micro Chemistry 10119) and AcOH (1 μL) is heated at 55° C. for 18 h. After cooling, the mixture is concentrated under reduced pressure and the residue is purified by FC on silica gel (CH₂Cl₂/acetone 97:3, then CH₂Cl₂/MeOH 97:3) to give the title compound as a colorless oil. RP-HPLC: t_{R}=5.45 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 563.4.

### Example 30

### (2S,4S,5S,7S)-5-Amino-4-hydroxy-2-isopropyl-7-[4-methoxy-3-(3-methoxy-propoxy)-benzyl]-8-methyl-nonanoic acid [(R) or (S)-1-(tetrahydro-furan-3-yl)methyl]-amide

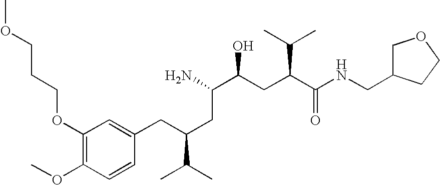

The solution of (2S,4S,5S,7S)-5-azido-4-hydroxy-2-isopropyl-7-[4-methoxy-3-(3-methoxy-propoxy)-benzyl]-8-methyl-nonanoic acid [3(R)- or 3(S)-(tetrahydro-furan-3-ylmethyl)-amide (406 mg, 0.721 mmol) in a 4:1-mixture of tert-butylmethylether and MeH (20 mL) is hydrogenated in the presence of Pd/C 10% (Engelhard 40708; 150 mg) and ethanolamine (0.048 mL) overnight at 25° C. under 1 atm. The mixture is filtered through Celite®, followed by washing with EtOH and evaporation of the combined filtrates to give the product its free base, which is dissolved in isopropanol (3.0 mL) and a 0.1 M solution of fumaric acid in isopropanol (3.28 mL) is added. The solvent is removed in vacuo and the residue is freeze-dried from dioxane to give the title compound as its hemi-fumarate salt (off-white powder). RP-HPLC: t_{R}=4.37 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 537.4.

### a) (2S,4S,5S,7S)-5-Azido-4-hydroxy-2-isopropyl-7-[4-methoxy-3-(3-methoxy-propoxy)-benzyl]-8-methyl-nonanoic acid [3(R)- or 3(S)-(tetrahydro-furan-3-ylmethyl)-amide

The title compound is obtained by chromatographic separation of the diastereomeric mixture of (2S,4S,5S,7S)-5-azido-4-hydroxy-2-isopropyl-7-[4-methoxy-3-(3-methoxy-propoxy)-benzyl]-8-methyl-nonanoic acid [3(R)- and 3(S)-(tetrahydro-furan-3-ylmethyl)-amide on a Chiralpak ADO preparative column (particle size: 20 μM; column dimensions: 5×20 cm; eluent: hexane/EtOH 9:1; flow rate 80 mL/min). Colorless oil. Diastereomeric purity de=99.4% by analytical chiral HPLC on Chiralpak AD-H® (column dimensions: 0.46×25 cm; eluent: n-hexane/EtOH 85:15; flow rate: 1 mL/min) with t_{R}=9.91 min (second eluting diastereomer). RP-HPLC: t_{R}=4.43 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 563.4.

### Example 31

### (2S,4S,5S,7S)-5-Amino-4-hydroxy-2-isopropyl-7-[4-methoxy-3-(2-methoxy-ethoxy)-benzyl]-8-methyl-nonanoic acid [(S)-1-(tetrahydro-furan-2-yl)methyl]-amide

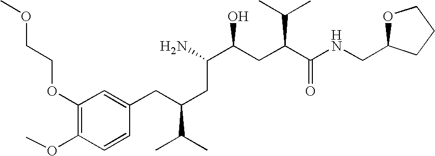

The title compound is prepared in a similar fashion as described in Example 27 from ((1S,2S,4S)-2-hydroxy-1-{(S)-2-[4-methoxy-3-(2-methoxy-ethoxy)-benzyl]-3-methyl-butyl}-5-methyl-4-{[(S)-1-(tetrahydro-furan-2-yl)methyl]-carbamoyl}-hexyl)-carbamic acid tert-butyl ester to afford, after freeze-drying from 4M HCl in dioxane, the hydrochloride salt as off-white powder. RP-HPLC: t_{R}=4.71 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 523.4.

The starting material is prepared as follows:

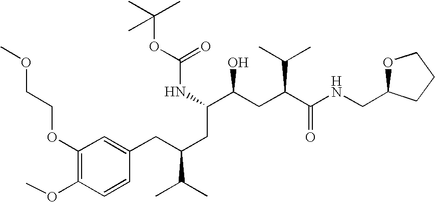

### a) ((1S,2S,4S)-2-Hydroxy-1-{(S)-2-[4-methoxy-3-(2-methoxy-ethoxy)-benzyl]-3-methyl-butyl}-5-methyl-4-{[(S)-1-(tetrahydro-furan-2-yl)methyl]-carbamoyl}-hexyl)-carbamic acid tert-butyl ester

The title compound is prepared as described in Example 27a) from {(1S,3S)-1-((S)-4-isopropyl-5-oxo-tetrahydro-furan-2-yl)-3-[3-(3-methoxy-propoxy)-4-methyl-benzyl]-4-methyl-pentyl}-carbamic acid tert-butyl ester (75 mg, 0.144 mmol) and [(S)-1-(tetrahydro-furan-2-yl)]-methylamine (150 μL, 1.44 mmol) in the presence of AcOH (0.5 mL). Colorless oil. RP-HPLC:

t_{R}=5.45 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 623.4.

### b) {(1S,3S)-1-((2S,4S)-4-Isopropyl-5-oxo-tetrahydro-furan-2-yl)-3-[3-(3-methoxy-ethoxy)-4-methyl-benzyl]-4-methyl-pentyl}-carbamic acid tert-butyl ester

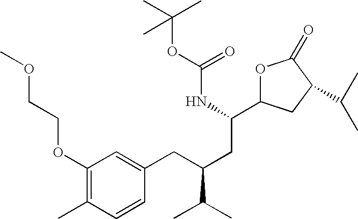

The title compound is prepared in a similar fashion as described in Example 26b) from (3S,5S)-5-{(1S,3S)-1-amino-3-[3-(3-methoxy-ethoxy)-4-methyl-benzyl]-4-methyl-pentyl}-3-isopropyl-dihydro-furan-2-one (2.53 g, 6.00 mmol), di-tert-butyl dicarbonate (4.58 g, 21.0 mmol) and N-ethyldiisopropylamine (4.11 mL, 24.0 mmol) in CH₂Cl₂ (50 mL). Pale yellow oil. RP-HPLC: t_{R}=5.99 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H₂O]⁺ 539.4.

### c) (3S,5S)-5-{(1S,3S)-1-Amino-3-[3-(3-methoxy-ethoxy)-4-methyl-benzyl]-4-methyl-pentyl}-3-isopropyl-dihydro-furan-2-one

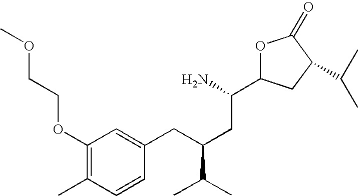

The title compound is prepared in a similar fashion as described in Example 26c) from isobutyric acid (S)-2-[(S)-2-azido-2-((2S,4S)-4-isopropyl-5-oxo-tetrahydro-furan-2-yl)-ethyl]-1-[3-(3-methoxy-ethoxy)-4-methyl-phenyl]-3-methyl-butyl ester (3.20 g, 6.00 mmol), dissolved in EtOH (300 mL), by hydrogenation over palladium on carbon (10%, 6.4 g; Engelhardt 40708) in the presence of ethanolamine (0.37 g, 6.00 mmol) at 25° C. under 1 atm for 24 hours. Yellowish oil. MS: [M+H]⁺ 422.2.

### d) Isobutyric acid (S)-2-[(S)-2-azido-2-((2S,4S)-4-isopropyl-5-oxo-tetrahydro-furan-2-yl)-ethyl]-1-[3-(3-methoxy-ethoxy)-4-methyl-phenyl]-3-methyl-butyl ester

The title compound is prepared in a similar fashion as described in Example 26d) from (3S,5S)-5-((1S,3S)-1-azido-3-{hydroxy-[3-(3-methoxy-ethoxy)-4-methyl-phenyl]-methyl}-4-methyl-pentyl)-3-isopropyl-dihydro-furan-2-one (2.82 g, 6.08 mmol), pyridine (2.45 mL, 30.4 mmol), 4-(N,N-dimethylamino)-pyridine (149 mg, 1.22 mmol) and isobutyric anhydride (3.03 mL, 18.5 mmol) in CH₂Cl₂ (60 mL), followed by FC purification on silica gel (gradient CH₂Cl₂ to CH₂Cl₂/acetone 98:2). Colorless oil. RP-HPLC: t_{R}=6.02 and 6.14 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H₂O]⁺551.4.

### e) (3S,5S)-5-((1S,3S)-1-Azido-3-{hydroxy-[3-(3-methoxy-ethoxy)-4-methyl-phenyl]-methyl}-4-methyl-Pentyl)-3-isopropyl-dihydro-furan-2-one

The title compound is prepared in a similar fashion as described in Example 26e) by the reaction of 4-bromo-1-methoxy-2-(2-methoxy-ethoxy)-benzene (2.50 g, 8.89 mmol), dissolved in dry THF (35 mL), with 1.6 M n-butyl lithium in hexane (10.0 mL, 16.0 mmol) in the presence of N-methylmorpholine (2.70 mL, 26.7 mmol), followed by trans-metallation with freshly prepared MgBr₂ (26.7 mmol; prepared from magnesium (650 mg) and 1,2-dibromoethane (2.30 mL) in 85 mL dry THF) and subsequent addition of (S)-2-[(S)-2-azido-2-((2S,4S)-4-isopropyl-5-oxo-tetrahydro-furan-2-yl)-ethyl]-3-methyl-butyraldehyde (2.50 g, 8.89 mmol) in THF (27 mL) over 20 min. The product is purified by flash chromatography on silica gel (CH₂Cl₂/acetone gradient 98:2 to 95:5) to afford the title compound as colorless oil. RP-HPLC: t_{R}=5.22 and 5.42 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H₂O]⁺481.3.

### f) 4-Bromo-1-methoxy-2-(2-methoxy-ethoxy)-benzene

The title compound is obtained as described for Example 26f) from 5-bromo-2-methyl-phenol (10.0 g, 49.3 mmol), 1-bromo-3-methoxy-propane (10.3 g, 73.9 mmol) and anhydrous K₂CO₃ (10.2 g, 73.9 mmol) in acetonitrile (200 mL). The crude product is dissolved in CH₂Cl₂, and the organics are washed with 0.1M NaOH, water and brine, dried over MgSO₄ and concentrated in vacuo. Yellowish oil. MS: [M+H₂O]⁺278.1/280.0.

### Example 32

### (2S,4S,5S,7S)-5-Amino-4-hydroxy-2-isopropyl-7-[4-methoxy-3-(2-methoxy-ethoxy)-benzyl]-8-methyl-nonanoic acid [(R)-1-(tetrahydro-furan-2-yl)methyl]-amide

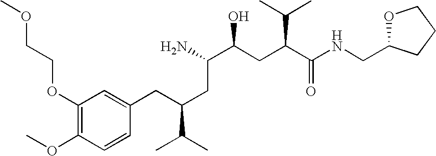

The title compound is prepared in a similar fashion as described in Example 27 from ((1S,2S,4S)-2-hydroxy-1-{(S)-2-[4-methoxy-3-(2-methoxy-ethoxy)-benzyl]-3-methyl-butyl}-5-methyl-4-{[(R)-1-(tetrahydro-furan-2-yl)methyl]-carbamoyl}-hexyl)-carbamic acid tert-butyl ester to afford its hemi-fumarate salt as off-white powder. RP-HPLC: t_{R}=4.70 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 523.4.

The starting material is prepared as follows:

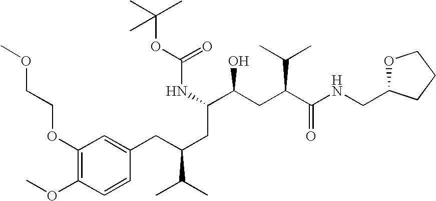

### a) ((1S,2S,4S)-2-Hydroxy-1-{(S)-2-[4-methoxy-3-(2-methoxy-ethoxy)-benzyl]-3-methyl-butyl}-5-methyl-4-{[(R)-1-(tetrahydro-furan-2-yl)methyl]-carbamoyl}-hexyl)-carbamic acid tert-butyl ester

The title compound is prepared as described in Example 27a) from {(1S,3S)-1-((S)-4-isopropyl-5-oxo-tetrahydro-furan-2-yl)-3-[3-(3-methoxy-propoxy)-4-methyl-benzyl]-4-methyl-pentyl}-carbamic acid tert-butyl ester (50 mg, 0.096 mmol) and [(R)-1-(tetrahydro-furan-2-yl)]-methylamine (100 μL, 0.96 mmol) in the presence of AcOH (0.5 μL). Colorless oil. RP-HPLC: t_{R}=5.47 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 623.4.

### Example 33

### (2S,4S,5S,7S)-5-Amino-4-hydroxy-2-isopropyl-7-[3-methoxy-5-(3-methoxy-propoxy)-benzyl]-8-methyl-nonanoic acid (tetrahydro-pyran-4-yl)-amide

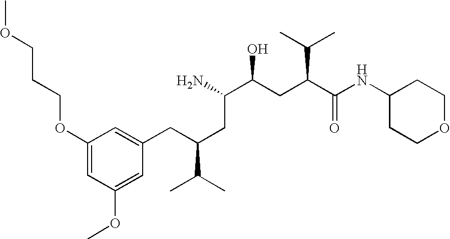

The title compound is prepared as described in Example 27 from [(1S,2S,4S)-2-hydroxy-1-{(S)-2-[3-methoxy-5-(3-methoxy-propoxy)-benzyl]-3-methyl-butyl}-5-methyl-4-(tetrahydro-pyran-4-ylcarbamoyl)-hexyl]-carbamic acid tert-butyl ester (110 mg, 0.173 mmol) by N—BOC deprotection with 4M HCl in dioxane (1.36 mL) and FC purification on silica gel ((CH₂Cl₂/MeOH/conc. NH₃ (10%) 93:7). The resulting free base is dissolved in isopropanol (1.0 mL) and a 0.1 M solution of fumaric acid in isopropanol (503 μL) is added. The solvent is removed in vacuo and the residue is freeze-dried to give the title compound as its hemi-fumarate salt (pale yellow powder). RP-HPLC: t_{R}=4.56 min (Nucleosil C-18HD column, 20-100% CH₃CN/H₂O/6 min, 100% CH₃CN/1.5 min, CH₃CN and H₂O containing 0.1% TFA, flow: 1.0 mL/min). MS: [M+H]⁺ 537.4.

The starting material is prepared as follows:

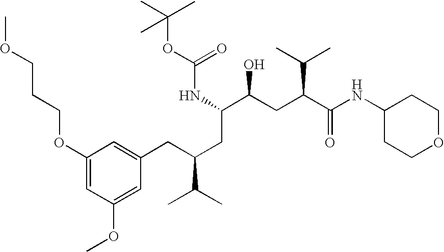

### a) [(1S,2S,4S)-2-Hydroxy-1-{(S)-2-[3-methoxy-5-(3-methoxy-propoxy)-benzyl]-3-methyl-butyl}-5-methyl-4-(tetrahydro-pyran-4-ylcarbamoyl)-hexyl]-carbamic acid tert-butyl ester

The title compound is prepared as described in Example 27a) from {(1S,3S)-1-((2S,4S)-4-isopropyl-5-oxo-tetrahydro-furan-2-yl)-3-[3-methoxy-5-(3-methoxy-propoxy)-benzyl]-4-methyl-pentyl}-carbamic acid tert-butyl ester (110 mg, 0.205 mmol) and tetrahydro-pyran-4-ylamine (207 mg, 2.05 mmol) in the presence of AcOH (0.5 μL). Purification by flash chromatography on silica gel (CH₂Cl₂/acetone 97:3 to 95:5) affords the product as colorless oil. RP-HPLC: t_{R}=5.63 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 637.4.

### b) {(1S,3S)-1-((2S,4S)-4-isopropyl-5-oxo-tetrahydro-furan-2-yl)-3-[3-methoxy-5-(3-methoxy propoxy)-benzyl]-4-methyl-pentyl}-carbamic acid tert-butyl ester

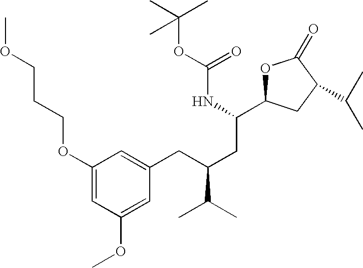

The title compound is prepared in a similar fashion as described in Example 26b) from (3S,5S)-5-{(1S,3S)-1-amino-3-[3-methoxy-5-(3-methoxy-propoxy)-benzyl]-4-methyl-pentyl}-3-isopropyl-dihydro-furan-2-one (2.36 g, 5.42 mmol), di-tert-butyl dicarbonate (4.14 g, 19.0 mmol) and N-ethyldiisopropylamine (3.71 mL, 21.7 mmol) in CH₂Cl₂ (55 mL). Purification by flash chromatography on silica gel (CH₂Cl₂/acetone gradient 100:0 to 98:2 to 95:5) affords the title compound as colorless oil. RP-HPLC: t_{R}=6.35 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H₂O]⁺553.4.

### c) (3S,5S)-5-{(1S,3S)-1-Amino-3-[3-methoxy-5-(3-methoxy-propoxy)-benzyl]-4-methyl-pentyl}-3-isopropyl-dihydro-furan-2-one

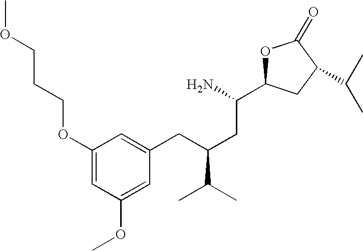

The title compound is prepared in a similar fashion as described in Example 26c) from isobutyric acid (S)-2-[(S)-2-azido-2-((2S,4S)-4-isopropyl-5-oxo-tetrahydro-furan-2-yl)-ethyl]-1-[3-methoxy-5-(3-methoxy-propoxy)-phenyl]-3-methyl-butyl ester (3.05 g, 5.57 mmol), dissolved in EtOH (300 mL), by hydrogenation over palladium on carbon (10%, 6.1 g; Engelhardt 40708) in the presence of ethanolamine (346 μL, 5.57 mmol) at 25° C. under 1 atm for 48 hours. Yellowish oil. RP-HPLC: t_{R}=5.31 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 436.2.

### d) Isobutyric acid (S)-2-[(S)-2-azido-2-((2S,4S)-4-isopropyl-5-oxo-tetrahydro-furan-2-yl)-ethyl]-1-[3-methoxy-5-(3-methoxy-propoxy)-phenyl]-3-methyl-butyl ester

The title compound is prepared in a similar fashion as described in Example 26d) from (3S,5S)-5-((1S,3S)-1-azido-3-{hydroxy-[3-methoxy-5-(3-methoxy-propoxy)-phenyl]-methyl}-4-methyl-pentyl)-3-isopropyl-dihydro-furan-2-one (2.72 g, 5.70 mmol), pyridine (2.29 mL, 28.5 mmol), 4-(N,N-dimethylamino)-pyridine (139 mg, 1.14 mmol) and isobutyric anhydride (2.84 mL, 17.1 mmol) in CH₂Cl₂ (55 mL), followed by aqueous extractive work-up and FC purification on silica gel (gradient CH₂Cl₂ to CH₂Cl₂/acetone 98:2). Colorless oil. RP-HPLC: t_{R}=6.41 and 6.53 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H₂O]⁺ 565.4.

### e) (3S,5S)-5-((1S,3S)-1-Azido-3-{hydroxy-[3-methoxy-5-(3-methoxy-propoxy)-phenyl]-methyl}-4-methyl-pentyl)-3-isopropyl-dihydro-furan-2-one

The title compound is prepared in a similar fashion as described in Example 26e) from 1-bromo-3-methoxy-5-(3-methoxy-propoxy)-benzene (2.47 g, 8.99 mmol) and (S)-2-[(S)-2-azido-2-((2S,4S)-4-isopropyl-5-oxo-tetrahydro-furan-2-yl)-ethyl]-3-methyl-butyraldehyde (2.30 g, 8.99 mmol) in the presence of N-methylmorpholine (2.7 mL, 24.5 mmol). After extractive work-up, the product is purified by flash chromatography on silica gel (CH₂Cl₂/acetone gradient 98:2 to 95:5) to afford the title compound as colorless oil. RP-HPLC: t_{R}=5.57 and 5.75 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H₂O]⁺495.2.

### f) 1-Bromo-3-methoxy-5-(3-methoxy-propoxy)-benzene

The title compound is obtained as described for Example 26f) from 3-Bromo-5-methoxy-phenol [855400-66-7] (2.4 g, 11.8 mmol), 1-bromo-3-methoxy-propane (2.71 g, 17.7 mmol) and anhydrous K₂CO₃ (2.45 g, 17.7 mmol) in acetonitrile (45 mL). The crude product is dissolved in CH₂Cl₂, and the organics are washed with 0.1 M NaOH, water and brine, dried over MgSO₄ and concentrated in vacuo. Oil. MS: [M]⁺275.0/277.0.

### Example 34

### (2S,4S,5S,7S)-5-Amino-4-hydroxy-2-isopropyl-7-[3-methoxy-5-(3-methoxy-propoxy)-benzyl]-8-methyl-nonanoic acid [(S)-1-(tetrahydro-furan-2-yl)methyl]-amide

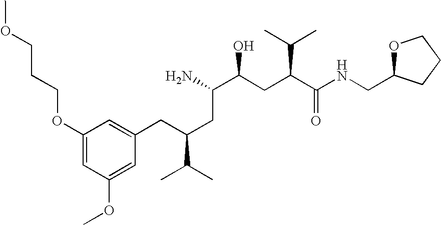

The title compound is prepared as described in Example 27 from ((1S,2S,4S)-2-hydroxy-1-{(S)-2-[3-methoxy-5-(3-methoxy-propoxy)-benzyl]-3-methyl-butyl}-5-methyl-4-{[(S)-1-(tetrahydro-furan-2-yl)methyl]-carbamoyl}-hexyl)-carbamic acid tert-butyl ester (125 mg, 0.196 mmol) by N—BOC deprotection with 4M HCl in dioxane (1.31 mL) and FC purification on silica gel (CH₂Cl₂/MeOH/conc. NH₃ (10%) 97:3). The resulting free base is dissolved in isopropanol (1.0 mL) and a 0.1 M solution of fumaric acid in isopropanol (765 μL) is added. The solvent is removed in vacuo and the residue is freeze-dried to give the title compound as its hemi-fumarate salt (off-white powder). RP-HPLC: t_{R}=4.71 min (Nucleosil C-18HD column, 20-100% CH₃CN/H₂O/6 min, 100% CH₃CN/1.5 min, CH₃CN and H₂O containing 0.1% TFA, flow: 1.0 mL/min). MS: [M+H]⁺ 537.4.

The starting material is prepared as follows:

### a) ((1S,2S,4S)-2-Hydroxy-1-{(S)-2-[3-methoxy-5-(3-methoxy-propoxy)-benzyl]-3-methyl-butyl}-5-methyl-4-{[(S)-1-(tetrahydro-furan-2-yl)methyl]-carbamoyl}-hexyl)-carbamic acid tert-butyl ester

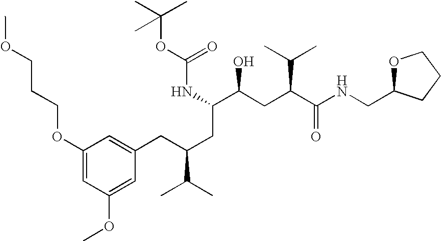

The title compound is prepared as described in Example 27a) from {(1S,3S)-1-((2S,4S)-4-isopropyl-5-oxo-tetrahydro-furan-2-yl)-3-[3-methoxy-5-(3-methoxy-propoxy)-benzyl]-4-methyl-pentyl}-carbamic acid tert-butyl ester (100 mg, 0.187 mmol) and [(S)-1-(tetrahydro-furan-2-yl)]-methylamine (193 μL, 1.87 mmol) in the presence of AcOH (0.5 μL). Purification by flash chromatography on silica gel (CH₂Cl₂/MeOH 97:3) affords the product as colorless oil. RP-HPLC: t_{R}=5.87 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 637.4.

### Example 35

### (2S,4S,5S,7S)-5-Amino-4-hydroxy-2-isopropyl-7-[3-methoxy-5-(3-methoxypropoxy)-benzyl]-8-methyl-nonanoic acid [(R)-1-(tetrahydro-furan-2-yl)methyl]-amide

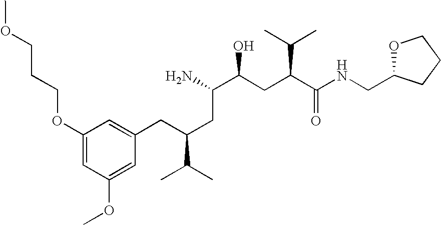

The title compound is prepared as described in Example 27 from ((1S,2S,4S)-2-hydroxy-1-{(S)-2-[3-methoxy-5-(3-methoxy-propoxy)-benzyl]-3-methyl-butyl}-5-methyl-4-{[(R)-1-(tetrahydro-furan-2-yl)methyl]-carbamoyl}-hexyl)-carbamic acid tert-butyl ester (107 mg, 0.168 mmol) by N—BOC deprotection with 4M HCl in dioxane (1.12 mL) and FC purification on silica gel (CH₂Cl₂/MeOH/conc. NH₃ (10%) 93:7). The resulting free base is dissolved in isopropanol (1.0 mL) and a 0.1 M solution of fumaric acid in isopropanol (475 μL) is added. The solvent is removed in vacuo and the residue is freeze-dried to give the title compound as its hemi-fumarate salt (off-white powder). RP-HPLC: t_{R}=4.69 min (Nucleosil C-18HD column, 20-100% CH₃CN/H₂O/6 min, 100% CH₃CN/1.5 min, CH₃CN and H₂O containing 0.1% TFA, flow: 1.0 mL/min). MS: [M+H]⁺ 537.4.

The starting material is prepared as follows:

### a) ((1S,2S,4S)-2-Hydroxy-1-{(S)-2-[3-methoxy-5-(3-methoxy-propoxy)-benzyl]-3-methyl-butyl}-5-methyl-4-{[(R)-1-(tetrahydro-furan-2-yl)methyl]-carbamoyl}-hexyl)-carbamic acid tert-butyl ester

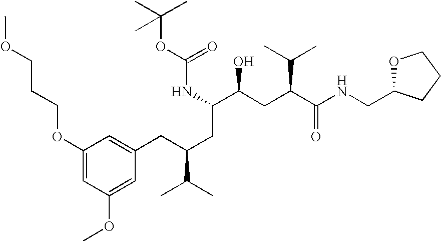

The title compound is prepared as described in Example 27a) from {(1S,3S)-1-((2S,4S)-4-isopropyl-5-oxo-tetrahydro-furan-2-yl)-3-[3-methoxy-5-(3-methoxy-propoxy)-benzyl]-4-methyl-pentyl}-carbamic acid tert-butyl ester (100 mg, 0.187 mmol) and [(R)-1-(tetrahydro-furan-2-yl)]-methylamine (193 μL, 1.87 mmol) in the presence of AcOH (0.5 μL). Purification by flash chromatography on silica gel (CH₂Cl₂/MeOH 97:3) affords the product as colorless oil. RP-HPLC: t_{R}=5.86 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 637.4.

### Example 36

### (2S,4S,5S,7S)-5-Amino-4-hydroxy-2-isopropyl-7-[4-methoxy-3-(4-methoxy-butyl)-benzyl]-8-methyl-nonanoic acid (tetrahydro-pyran-4-yl)-amide

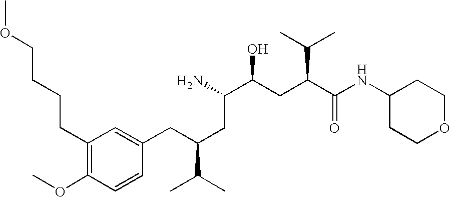

The title compound is prepared using the protocol described in Example 22 starting from (2S,4S,5S,7S)-5-azido-4-hydroxy-2-isopropyl-7-[4-methoxy-3-(4-methoxy-butyl)-benzyl]-8-methyl-nonanoic acid (tetrahydro-pyran-4-yl)-amide acid (tetrahydro-pyran-4-yl)-amide (260 mg, 0.464 mmol). The crude product is purified by FC on silica gel (CH₂Cl₂/MeOH/NH₃ conc. (10%) 97:3, then CH₂Cl₂/MeOH/NH₃ conc. (10%) 85:15) to give the title compound as its free base, which is dissolved in isopropanol (3.0 mL) and a 0.1 M solution of fumaric acid in isopropanol (2.11 mL) is added. The solvent is removed in vacuo and the residue is freeze-dried from dioxane to give the title compound as its hemi-fumarate salt (off-white powder). RP-HPLC: t_{R}=4.90 min (Nucleosil C18HD column, 5-100% CH₃CN/H₂O/6 min, 100% CH₃CN/1.5 min, CH₃CN and H₂O containing 0.1% TFA, flow: 1.0 mL/min). MS: [M+H]⁺ 535.4.

The starting material is prepared as follows:

### a) (2S,4S,5S,7S)-5-Azido-4-hydroxy-2-isopropyl-7-[4-methoxy-3-(4-methoxy-butyl)-benzyl]-8-methyl-nonanoic acid (tetrahydro-pyran-4-yl)-amide acid (tetrahydro-pyran-4-yl)-amide

The title compound is prepared from (S)-5-{(1S,3S)-1-azido-3-[4-methoxy-3-(4-methoxy-butyl)-benzyl]-4-methyl-pentyl}-3-isopropyl-dihydro-furan-2-one (225 mg, 0.49 mmol; prepared as described in EP678503A1) and tetrahydro-pyran-4-ylamine (496 mg, 4.9 mmol) as described in Example 22a) and purification by FC on silica gel (CH₂Cl₂/acetone 97:3, then CH₂Cl₂/MeOH 95:5). Yellowish oil. RP-HPLC: t_{R}=6.26 min (Nucleosil 100-5 C18 column, 10-100% CH₃CN/H₂O/5 min, 100% CH₃CN/2.5 min, CH₃CN and H₂O containing 0.1% TFA, flow: 1.0 mL/min). MS: [M+H]⁺ 561.4.

### Example 37

### Film-Coated Tablets

The following constituents are processed for the preparation of 10 000 tablets each containing 100 mg of active ingredient:

| | |
|---|---|
| active ingredient | 1000 g |
| corn starch | 680 g |
| colloidal silicic acid | 200 g |
| magnesium stearate | 20 g |
| stearic acid | 50 g |
| sodium carboxymethyl starch | 250 g |
| water | quantum satis |

A mixture of one of the compounds of formula (I), mentioned in the preceding Examples, as active ingredient, 50 g of corn starch and the colloidal silicic acid is processed into a moist mass with starch paste prepared from 250 g of corn starch and 2.2 kg of demineralised water. The mass is forced through a sieve having a mesh size of 3 mm and dried at 45° C. for 30 minutes in a fluidised bed drier. The dried granules are pressed through a sieve having a mesh size of 1 ram, mixed with a previously sieved mixture (1 mm sieve) of 330 g of corn starch, the magnesium stearate, the stearic acid and the sodium carboxymethyl starch and compressed to form slightly biconvex tablets.

Although the present invention has been described in considerable detail with reference to certain preferred versions thereof, other versions are possible without departing from the spirit and scope of the preferred versions contained herein. All references and Patents (U.S. and others) referred to herein are hereby incorporated by reference in their entirety as if set forth in full herein.

### Biological Tests

Renin inhibitory activity was assessed in vitro by FRET assay (as above described). Results for representative compounds of formula I are:

| Compound | IC₅₀, nM (hu-renin FRET assay) |
|---|---|
| Example 1 | 1.4 |
| Example 2 | 4.0 |
| Example 3 | 0.7 |
| Example 5 | 3.2 |
| Example 10 | 3.5 |
| Example 11 | 4.0 |
| Example 12 | 0.7 |
| Example 13 | 0.2 |
| Example 14 | 1.4 |
| Example 17 | 2.5 |
| Example 18 | 0.9 |
| Example 19 | 8.0 |
| Example 20 | 7.0 |
| Example 22 | 2.5 |
| Example 23 | 0.5 |
| Example 26 | 0.5 |
| Example 27 | 0.5 |
| Example 28 | 0.6 |
| Example 29 | 0.4 |
| Example 30 | 0.8 |
| Example 36 | 0.3 |

We claim:

1. A 6-amino-γ-hydroxy-ω-aryl-alkanoic acid amide compound of formula (I) 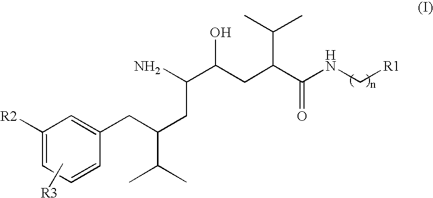
wherein
R1 is
a) oxetanyl, tetrahydrofuranyl, tetrahydropyranyl, furanyl, dioxolanyl or dioxanyl, each of which is optionally substituted, one or more times, by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl, nitro, or oxo; or
b) piperidyl which is substituted, one or more times, by C₂₋₆alkenyl, C₂₋₆alkynyl, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl or nitro; which is bonded via a C atom; or
c) pyrrolidinyl which is substituted, one or more times, by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro; which is bonded via a C atom; or
d) bicyclic saturated heterocyclyl which is optionally substituted, one or more times, by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro; which is bonded via a C atom; and
R2 and R3, independently of one another, are selected from C₁₋₈alkyl, C₁₋₈alkoxy, C₁₋₄alkoxy-C₁₋₄alkoxy, C₁₋₄alkoxy-C₁₋₄alkyl, halo-C₁₋₈alkoxy, halo-C₁₋₈alkyl, C₁₋₈alkanoyl, C₃₋₈cycloalkyl or halogen; and
n is 0 or 1;
or a salt thereof.

2. The compound according to claim 1 of the formula (III) 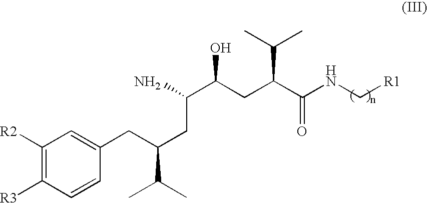
wherein
R1 is
a) oxetanyl, tetrahydrofuranyl, tetrahydropyranyl, furanyl, dioxolanyl or dioxanyl, each of which is optionally substituted, one or more times, by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl, nitro, or oxo; or
b) piperidyl which is substituted, one or more times, by C₂₋₆alkenyl, C₂₋₆alkynyl, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl or nitro; which is bonded via a C atom; or
c) pyrrolidinyl which is substituted, one or more times, by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro; which is bonded via a C atom; or
d) bicyclic saturated heterocyclyl which is optionally substituted, one or more times, by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro; which is bonded via a C atom; and
R2 and R3, independently of one another, are selected from C₁₋₈alkyl, C₁₋₈alkoxy, C₁₋₄alkoxy-C₁₋₄alkoxy, C₁₋₄alkoxy-C₁₋₄alkyl, halo-C₁₋₈alkoxy, halo-C₁₋₈alkyl, C₁₋₈alkanoyl, C₃₋₈cycloalkyl or halogen; and
n is 0 or 1;
or a salt thereof.

3. The compound according to claim 1 wherein R2 and R3, independently of one another, are selected from C₁₋₈alkyl, C₁₋₈alkoxy, C₁₋₄alkoxy-C₁₋₄alkoxy or C₁₋₄alkoxy-C₁₋₄alkyl;
or a salt thereof.

4. The compound according to claim 1, wherein n is 0; or a salt thereof.

5. The compound according to claim 1, wherein n is 1 and R1 is selected from oxetanyl, tetrahydrofuranyl, tetrahydropyranyl, furanyl, dioxolanyl or dioxanyl, each of which is optionally substituted, one or more times, by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl, nitro, or oxo; or salt thereof.

6. The compound according to claim 1 wherein R1 is oxetanyl, tetrahydrofuranyl, tetrahydropyranyl, furanyl, dioxolanyl or dioxanyl, each of which is optionally substituted, one or more times, by C₁₋₆alkoxy, C₁₋₆alkyl, aryl-C₁₋₆alkyl, optionally esterified carboxy, C₃₋₈cycloalkyl-C₀₋₆alkyl, hydroxyl or oxo;
or a salt thereof.

7. The compound according to claim 1, wherein R1 is oxetanyl, tetrahydrofuranyl, tetrahydropyranyl, furanyl, dioxolanyl or dioxanyl, each of which is optionally substituted, one or more times, by C₁₋₆alkyl, hydroxyl or oxo;
or a salt thereof.

8. The compound according to claim 1, wherein R1 is piperidyl which is substituted, one or more times, by C₁₋₆alkyl, aryl-C₁₋₆alkyl, optionally esterified carboxy or C₃₋₈cycloalkyl-C₀₋₆alkyl; which is bonded via a C atom;
or a salt thereof.

9. The compound according to claim 1, wherein R1 is piperidyl which is substituted, one or more times, by C₁₋₆alkyl, aryl-C₁₋₆alkyl or optionally esterified carboxy; which is bonded via a C atom;
or a salt thereof.

10. The compound according to claim 1, wherein R1 is pyrrolidinyl, which is substituted, one or more times, by C₁₋₆alkoxy, C₁₋₆alkyl, aryl-C₁₋₆alkyl, optionally esterified carboxy, C₃₋₈cycloalkyl-C₀₋₆alkyl or hydroxyl; which is bonded via a C atom;
or a salt thereof.

11. The compound according to claim 1, wherein R1 is pyrrolidinyl, which is substituted, one or more times, by C₁₋₆alkyl, aryl-C₁₋₆alkyl, optionally esterified carboxy or hydroxyl; which is bonded via a C atom;
or a salt thereof.

12. The compound according to claim 1, wherein R1 is bicyclic saturated heterocyclyl which is optionally substituted, one or more times, by C₁₋₆alkoxy, C₁₋₆alkyl, aryl-C₁₋₆alkyl, optionally esterified carboxy, C₃₋₈cycloalkyl-C₀₋₆alkyl, hydroxyl or oxo; or salt thereof.

13. The compound according to claim 1, wherein R1 is bicyclic saturated heterocyclyl which is optionally substituted, one or more times, by C₁₋₆alkyl, aryl-C₁₋₆alkyl, optionally esterified carboxy, hydroxyl or oxo; or a salt thereof.

14. The compound according to claim 12, wherein the bicyclic saturated heterocyclic radical is unsubstituted and comprises a nitrogen atom as heretoatom; or a salt thereof.

15. The compound according to claim 1, wherein R2 is C₁₋₄alkoxy-C₁₋₄alkoxy, and R3 is C₁₋₈alkyl or C₁₋₈alkoxy; or a salt thereof.

16. The compound according to claim 1, wherein R2 is 3-methoxypropyloxy and R3 is methoxy or methyl; or a salt thereof.

17. The compound according to claim 1, wherein R2 is 2-methoxyethyloxy and R3 is methoxy; or a salt thereof.

18. The compound according to claim 1, wherein R2 is C₁₋₄alkoxy-C₁₋₄alkyl, and R3 is C₁₋₈alkyl or C₁₋₈alkoxy.

19. The compound according to claim 1, wherein R2 is 4-methoxybutyl and R3 is methoxy; or a salt thereof.

20. The compound of formula (I) according to claim 1 selected from the group consisting of:
(2S,4S,5S,7S)-5-Amino-4-hydroxy-2-isopropyl-7-[4-methoxy-3-(3-methoxy-propoxy)-benzyl]-8-methyl-nonanoic acid (tetrahydro-pyran-4-yl)-amide;
(2S,4S,5S,7S)-5-Amino-4-hydroxy-2-isopropyl-7-[4-methoxy-3-(3-methoxy-propoxy)-benzyl]-8-methyl-nonanoic acid ((R)-1-methyl-pyrrolidin-3-yl)-amide;
(2S,4S,5S,7S)-5-Amino-4-hydroxy-2-isopropyl-7-[4-methoxy-3-(3-methoxy-propoxy)-benzyl]-8-methyl-nonanoic acid ((S)-1-methyl-pyrrolidin-3-yl)-amide;
(2S,4S,5S,7S)-5-Amino-4-hydroxy-2-isopropyl-7-[4-methoxy-3-(3-methoxy-propoxy)-benzyl]-8-methyl-nonanoic acid (1-aza-bicyclo[2.2.2]oct-3-yl)-amide;
(2S,4S,5S,7S)-5-Amino-4-hydroxy-2-isopropyl-7-[4-methoxy-3-(3-methoxy-propoxy)-benzyl]-8-methyl-nonanoic acid (1-methyl-piperidin-4-yl)-amide;
(2S,4S,5S,7S)-5-Amino-4-hydroxy-2-isopropyl-7-[4-methoxy-3-(3-methoxy-propoxy)-benzyl]-8-methyl-nonanoic acid (1-benzyl-piperidin-4-yl)-amide;
4-{(2S,4S,5S,7S)-5-Amino-4-hydroxy-2-isopropyl-7-[4-methoxy-3-(3-methoxy-propoxy)-benzyl]-8-methyl-nonanoylamino}-piperidine-1-carboxylic acid tert-butyl ester;
(2S,4S,5S,7S)-5-Amino-4-hydroxy-2-isopropyl-7-[4-methoxy-3-(3-methoxy-propoxy)-benzyl]-8-methyl-nonanoic acid ((S)-2-oxo-tetrahydro-furan-3-yl)-amide;
(2S,4S,5S,7S)-5-Amino-4-hydroxy-2-isopropyl-7-[4-methoxy-3-(3-methoxy-propoxy)-benzyl]-8-methyl-nonanoic acid (3-methyl-oxetan-3-ylmethyl)-amide;
(2S,4S,5S,7S)-5-Amino-4-hydroxy-2-isopropyl-7-[4-methoxy-3-(3-methoxy-propoxy)-benzyl]-8-methyl-nonanoic acid [(S)-1-(tetrahydro-furan-2-yl)methyl]-amide;
(2S,4S,5S,7S)-5-Amino-4-hydroxy-2-isopropyl-7-[4-methoxy-3-(3-methoxy-propoxy)-benzyl]-8-methyl-nonanoic acid [(R)-1-(tetrahydro-furan-2-yl)methyl]-amide;
(2S,4S,5S,7S)-5-Amino-4-hydroxy-2-isopropyl-7-[4-methoxy-3-(3-methoxy-propoxy)-benzyl]-8-methyl-nonanoic acid ((R)-tetrahydrofuran-3-yl)-amide;
(2S,4S,5S,7S)-5-Amino-4-hydroxy-2-isopropyl-7-[4-methoxy-3-(3-methoxy-propoxy)-benzyl]-8-methyl-nonanoic acid ((S)-tetrahydrofuran-3-yl)-amide;
(2S,4S,5S,7S)-5-Amino-4-hydroxy-2-isopropyl-7-[4-methoxy-3-(3-methoxy-propoxy)-benzyl]-8-methyl-nonanoic acid (tetrahydro-pyran-4-ylmethyl)-amide;
(2S,4S,5S,7S)-5-Amino-4-hydroxy-2-isopropyl-7-[4-methoxy-3-(3-methoxy-propoxy)-benzyl]-8-methyl-nonanoic acid ([1,3]dioxolan-2-ylmethyl)-amide;
(2S,4S,5S,7S)-5-Amino-4-hydroxy-2-isopropyl-7-[4-methoxy-3-(3-methoxy-propoxy)-benzyl]-8-methyl-nonanoic acid (5,5-dimethyl-[1,3]dioxan-2-ylmethyl)-amide;
(2S,4S,5S,7S)-5-Amino-4-hydroxy-2-isopropyl-7-[4-methoxy-3-(3-methoxy-propoxy)-benzyl]-8-methyl-nonanoic acid (furan-3-ylmethyl)-amide;
(2S,4S,5S,7S)-5-Amino-4-hydroxy-2-isopropyl-7-[4-methoxy-3-(3-methoxy-propoxy)-benzyl]-8-methyl-nonanoic acid (furan-2-ylmethyl)-amide;
(2S,4S,5S,7S)-5-Amino-4-hydroxy-2-isopropyl-7-[4-methoxy-3-(3-methoxy-propoxy)-benzyl]-8-methyl-nonanoic acid [2(R) or 2(S)-1-(tetrahydro-pyran-2-yl)methyl]-amide;
(2S,4S,5S,7S)-5-Amino-4-hydroxy-2-isopropyl-7-[4-methoxy-3-(3-methoxy-propoxy)-benzyl]-8-methyl-nonanoic acid (4-hydroxy-tetrahydro-pyran-4-ylmethyl)-amide;
(2S,4S,5S,7S)-5-Amino-4-hydroxy-2-isopropyl-7-[4-methoxy-3-(3-methoxy-propoxy)-benzyl]-8-methyl-nonanoic acid [(R)- or (S)-3-(tetrahydro-pyran-3-yl)]-amide;
(2S,4S,5S,7S)-5-Amino-4-hydroxy-2-isopropyl-7-[4-methoxy-3-(3-methoxy-propoxy)-benzyl]-8-methyl-nonanoic acid [trans-(3S,4R)- or [(3R,4S)-4-hydroxy-tetrahydro-furan-3-yl]-amide;
(2S,4S,5S,7S)-5-Amino-4-hydroxy-2-isopropyl-7-[3-(3-methoxy-propoxy)-4-methyl-benzyl]-8-methyl-nonanoic acid (tetrahydro-pyran-4-yl)-amide;
(2S,4S,5S,7S)-5-Amino-4-hydroxy-2-isopropyl-7-[3-(3-methoxy-propoxy)-4-methyl-benzyl]-8-methyl-nonanoic acid [(R)-1-(tetrahydro-furan-2-yl)methyl]-amide;
(2S,4S,5S,7S)-5-Amino-4-hydroxy-2-isopropyl-7-[3-(3-methoxy-propoxy)-4-methyl-benzyl]-8-methyl-nonanoic acid [(S)-1-(tetrahydro-furan-2-yl)methyl]-amide;
(2S,4S,5S,7S)-5-Amino-4-hydroxy-2-isopropyl-7-[4-methoxy-3-(3-methoxy-propoxy)-benzyl]-8-methyl-nonanoic acid [3(R) or 3(S)-1-(tetrahydro-furan-3-yl)methyl]-amide;
(2S,4S,5S,7S)-5-Amino-4-hydroxy-2-isopropyl-7-[4-methoxy-3-(2-methoxy-ethoxy)-benzyl]-8-methyl-nonanoic acid [(S)-1-(tetrahydro-furan-2-yl)methyl]-amide;
(2S,4S,5S,7S)-5-Amino-4-hydroxy-2-isopropyl-7-[4-methoxy-3-(2-methoxy-ethoxy)-benzyl]-8-methyl-nonanoic acid [(R)-1-(tetrahydro-furan-2-yl)methyl]-amide;
(2S,4S,5S,7S)-5-Amino-4-hydroxy-2-isopropyl-7-[3-methoxy-5-(3-methoxy-propoxy)-benzyl]-8-methyl-nonanoic acid (tetrahydro-pyran-4-yl)-amide;
(2S,4S,5S,7S)-5-Amino-4-hydroxy-2-isopropyl-7-[3-methoxy-5-(3-methoxy-propoxy)-benzyl]-8-methyl-nonanoic acid [(S)-1-(tetrahydro-furan-2-yl)methyl]-amide;
(2S,4S,5S,7S)-5-Amino-4-hydroxy-2-isopropyl-7-[3-methoxy-5-(3-methoxypropoxy)-benzyl]-8-methyl-nonanoic acid [(R)-1-(tetrahydro-furan-2-yl)methyl]-amide and
S,4S,5S,7S)-5-Amino-4-hydroxy-2-isopropyl-7-[4-methoxy-3-(4-methoxy-butyl)-benzyl]-8-methyl-nonanoic acid (tetrahydro-pyran-4-yl)-amide.

21. A method for the treatment of hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders, comprising:
administering a therapeutically effective amount of a compound, to a warm-blooded animal in need thereof,
wherein the compound is a 6-amino-γ-hydroxy-ω-aryl-alkanoic acid amide compound of formula (I) 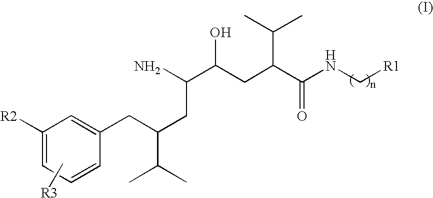
wherein
R1 is
a) oxetanyl, tetrahydrofuranyl, tetrahydropyranyl, furanyl, dioxolanyl or dioxanyl, each of which is optionally substituted, one or more times, by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl, nitro, or oxo; or
b) piperidyl which is substituted, one or more times, by C₂₋₆alkenyl, C₂₋₆alkynyl, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl or nitro; which is bonded via a C atom; or
c) pyrrolidinyl which is substituted, one or more times, by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro; which is bonded via a C atom; or
d) bicyclic saturated heterocyclyl which is optionally substituted, one or more times, by C₂₋₆alkenyl, C₂₋₆alkynyl, C₁₋₆alkoxy, C₁₋₆alkoxy-C₁₋₆alkoxy, C₁₋₆alkoxy-C₁₋₆alkyl, C₁₋₆alkoxycarbonylamino, C₁₋₆alkyl, C₀₋₆alkylcarbonylamino, C₁₋₆alkylcarbonyloxy, C₁₋₆alkylenedioxy, unsubstituted or N-mono or N,N-di-C₁₋₆alkylated amino, aryl, aryl-C₁₋₆alkyl, unsubstituted or N-mono or N,N-di-C₁₋₆alkylated carbamoyl, optionally esterified carboxy, cyano, C₃₋₈cycloalkoxy, C₃₋₈cycloalkyl-C₀₋₆alkyl, halogen, halo-C₁₋₆alkoxy, halo-C₁₋₆alkyl, heteroaryl, unsaturated or partially saturated or saturated heterocyclyl, hydroxyl or nitro; which is bonded via a C atom; and
R2 and R3, independently of one another, are selected from C₁₋₈alkyl, C₁₋₈alkoxy, C₁₋₄alkoxy-C₁₋₄alkoxy, C₁₋₄alkoxy-C₁₋₄alkyl, halo-C₁₋₈alkoxy, halo-C₁₋₈alkyl, C₁₋₈alkanoyl, C₃₋₈cycloalkyl or halogen; and
n is 0 or 1;
or a salt thereof.

22. A pharmaceutical composition, comprising:
the compound according to claim 1 and
one or more pharmaceutically acceptable excipient(s).

23. A pharmaceutical composition according to claim 22, comprising:
a therapeutically effective amount of the compound in combination with a therapeutically effective amount of an anti-diabetic agent, a hypolipidemic agent, an anti-obesity agent or an anti-hypertensive agent.
